(19) United States
(12) Patent Application Publication
(10) Pub. No.: US 2009/0208960 A1
(43) Pub. Date: Aug. 20, 2009

(54) METHODS FOR IDENTIFYNG CELLULAR MODULATORS OF DISAGGREGATION ACTIVITY OR AGGREGATION ACTIVITY IN AN ANIMAL

(76) Inventors: Jeffrery W. Kelly, La Jolla, CA (US); Jan Gerd Maximilian Bieschke, Berlin (DE); Andrew Dillin, La Jolla, CA (US); Ehud Cohen, San Diego, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 12/368,924

(22) Filed: Feb. 10, 2009

Related U.S. Application Data
(63) Continuation of application No. PCT/US2007/075722, filed on Aug. 10, 2007.
(60) Provisional application No. 60/837,096, filed on Aug. 10, 2006.

(51) Int. Cl. C12Q 1/68; C12Q 1/00

(52) U.S. Cl. 435/6; 435/4

(57) ABSTRACT

Methods for identifying a cellular modulator of a biological disaggregation activity or a biological aggregation activity of an animal are provided. Methods for identifying a compound which modulates biological disaggregation activity or a biological aggregation activity in a biological sample are provided.

### CROSS REFERENCE TO RELATED APPLICATION

This is a continuation of International Application No. PCT/US2007/075722, with an international filing date of Aug. 10, 2007, which claims the benefit of U.S. Provisional Application No. 60/837,096, filed Aug. 10, 2006, both of which are hereby incorporated by reference in their entirety.

### STATEMENT OF GOVERNMENT SUPPORT

This invention was made by government support by Grant No. DK46335 and NS50636 from National Institutes of Health. The Government has certain rights in this invention.

### FIELD

The present invention generally relates to methods for assaying aggregation and disaggregation of macromolecules to discover molecules regulated by the aging program and that ameliorate proteotoxicity and neurodegeneration associated with Alzheimer's disease, Parkinson's disease, Huntington's disease and related diseases.

### BACKGROUND

Late onset human neurodegenerative diseases including Alzheimer's (AD), Huntington's, and Parkinson's diseases are genetically and pathologically linked to aberrant protein aggregation. Selkoe, Nature 426: 900, 2003; Kopito and Ron, Nat Cell Biol 2: E207, 2000. In AD, formation of aggregation-prone peptides, particularly Aβ₁₋₄₂, by endoproteolysis of the Amyloid Precursor Protein (APP) is associated with the disease through an unknown mechanism. Bossy-Wetzel et al., Nat Med 10 Suppl, S2, 2004; Caughey and Lansbury, Annu Rev Neurosci 26:267, 2003. Whether intracellular accumulation or extracellular deposition of Aβ₁₋₄₂ initiates the pathological process is a key unanswered question. Takahashi et al., Am J. Pathol 161: 1869, 2002. Typically, individuals who carry AD-linked mutations present with clinical symptoms during their fifth or sixth decade, while sporadic cases appear after the seventh decade. Why aggregation-mediated toxicity emerges late in life and whether it is mechanistically linked to the aging process remains unclear.

Perhaps the most prominent pathway that regulates lifespan and youthfulness in worms, flies, and mammals is the insulin/insulin-like signaling (IIS) pathway. Kenyon, Cell 120: 449, 2005. In the nematode Caenorhabditis elegans, the sole insulin/IGF-1 receptor, DAF-2 (Kimura et al., Science 277: 942, 1997), initiates the transduction of a signal that causes the phosphorylation of the FOXO transcription factor, DAF-16 (Lin et al., Science 278: 1319, 1997; Ogg et al., Nature 389: 994, 1997), preventing its translocation to the nucleus. Lin et al., Nat Genet 28: 139, 2001. This negative regulation of DAF-16 compromises expression of its target genes, decreases stress resistance, and shortens the worm's lifespan. Thus, knockdown of daf-2 expression creates long-lived, youthful, stress-resistant worms. Kenyon et al., Nature 366: 461, 1993. Similarly, suppression of the mouse DAF-2 ortholog, IGF 1-R, creates long-lived mice. Holzenberger et al., Nature 421: 182, 2003. Recent studies indicate that in worms, life-span extension due to reduced daf-2 activity is also dependent upon the Heat Shock Factor 1 (HSF-1). Moreover, increased expression of hsf-1 extends worm lifespan in a daf-16 dependent manner. Hsu et al., Science 300:1142, 2003. That the DAF-16 and HSF-1 transcriptomes result in the expression of numerous chaperones (Hsu et al., Science 300: 1142, 2003; Morley and Morimoto, Mol Biol Cell 15: 657, 2004) suggest that the integrity of protein folding could play a role in lifespan determination and the amelioration of aggregation-associated proteotoxicity. Indeed, amelioration of Huntington-associated proteotoxicity by slowing the aging process in worms has been reported. Hsu et al., Science 300: 1142, 2003; Morley et al., Proc Natl Acad Sci USA 99: 10417, 2002; Parker et al., Nat Genet 37: 349, 2005. Studies have provided methods for diagnostic detection of diseases associated with pathological protein deposition. U.S. Pat. No. 6,498,017; U.S. Pat. No. 5,948,763; U.S. Pat. No. 6,689,753; PCT International Application WO 2002073210; PCT International Application WO 9915903; Castilla et al., Nature Medicine, 11: 982-985, 2005; Saa, et al., Science, 313: 92-94, 2006.

A need exists in the art for improved kinetic disaggregation and aggregation assays to discover molecules regulated by the aging program and identified by biological aggregation and disaggregation activities and compounds that alter the biological aggregation and disaggregation activities.

### SUMMARY

Methods for assaying aggregation and disaggregation of macromolecules are provided to discover molecules regulated by the aging program and that ameliorate proteotoxicity and neurodegeneration associated with disease, for example, Alzheimer's disease, Parkinson's disease, Huntington's disease and related diseases. Method for identifying therapeutic compounds that are useful for treatment of disease are provided, wherein the methods utilize an assay for aggregation and disaggregation of macromolecules. The assays can be used to screen compounds to discover therapeutic compounds that interact with biological molecules that are regulated by the aging program and that ameliorate proteotoxicity and neurodegeneration associated with disease, for example, Alzheimer's disease, Parkinson's disease, Huntington's disease and related diseases.

A method for identifying a cellular modulator of a biological disaggregation activity of an animal is provided which comprises, providing one or more polypeptide aggregate fibrils in solution, contacting a biological sample with the polypeptide aggregate fibrils, measuring a rate of fibril disappearance, and identifying the modulator within the biological sample from the rate of fibril disappearance in the biological disaggregation activity. The method can further comprise forming the one or more polypeptide aggregate fibrils by transforming an amyloidogenic polypeptide or analog thereof into the one or more polypeptide aggregate fibrils. Alternatively the one or more polypeptide aggregate fibrils can be derived from a biological cell or tissue. In one aspect, the method further comprises transforming with seeding an amyloidogenic polypeptide or analog thereof into a polypeptide aggregate fibrin. In another aspect, the method further comprises measuring the rate of fibril disappearance in the presence of at least one protease inhibitor The biological disaggregation activity can be denaturable. The cellular modulator can be located within an intracellular fraction or an extracellular fraction. The polypeptide aggregate fibril includes, but is not limited to, amyloid fibrils, alpha synuclein aggregate fibrils, or polyglutamine aggregate fibrils. The polypeptide aggregate fibrils further include immunoglobulin light chain aggregate fibrils and trans-thyretin. The rate of fibril disappearance can be measured by a reduction in amyloid fibril aggregates. The biological sample can be from an animal with a mutation causing an aging program perturbation. The mutation causing the aging program perturbation can be in daf-2, daf-16, or hsf-1 in Caenorhabditis elegans. In one aspect, the cellular modulator detoxifies polypeptide aggregate fibrils regulated by an aging program in the animal. In a further aspect, the cellular modulator detoxifies protein aggregates not regulated by an aging program in the animal.

In the methods provided herein, measuring the rate of fibril disappearance can be performed utilizing a number of techniques. In one aspect, the method further comprises measuring the rate of fibril disappearance by measuring a reduction in fluorescence of fibril-binding environment sensitive fluorophores. In a detailed aspect, the fluorophores exhibit stronger fluorescence or wavelength-shifted fluorescence, or a combination thereof, when bound to fibrils compared to when the fluorophores are solvated in aqueous medium. The fluorophore includes, but is not limited to, thioflavin T/S or Congo red. In a further aspect, the method comprises measuring the rate of fibril disappearance by atomic force microscopy, electron microscopy or light microscopy. In a further aspect, the method comprises measuring the rate of fibril disappearance by an decrease in anisotropy of fluorescently labeled solubilized amyloidogenic peptides. The method further comprises measuring the rate of fibril disappearance by a decrease in turbidity or light scattering of the biological sample. The method further comprises measuring the rate of fibril disappearance by appearance of monomers or low molecular weight oligomers of amyloid fibrils. The method further comprises measuring the appearance of monomers or low molecular weight oligomers is detected by gel electrophoresis, spectroscopically, chromomatographically, mass spectrometry or liquid chromatography mass spectrometry (LCMS). The method further comprises measuring a reduction of aggregates by SDS polyacrylamide gel electrophoresis followed by Western blotting. The method further comprises measuring a reduction of aggregates by sucrose gradient centrifugation. The method further comprises measuring a reduction of aggregates by native gel electrophoresis visualized by antibodies or amyloidophilic dyes.

A method for identifying a compound which modulates biological disaggregation activity in an animal is provided which comprises contacting a polypeptide aggregate fibril with the compound, providing a biological sample from the animal in an amount selected to be effective to modulate biological disaggregation activity, measuring a rate of fibril disappearance in the presence of the compound compared to a rate of fibril disappearance in the absence of the compound, detecting an effect of the compound on the biological disaggregation activity, effectiveness of the compound being indicative of an increase in biological disaggregation activity. In one aspect, the polypeptide aggregate fibril is a labeled polypeptide aggregate fibril prepared in vitro. The label can be a fluorophore. The label includes, but is not limited to, thioflavin T/S or Congo red. The polypeptide aggregate fibril can be derived from a biological source. In one aspect, the method further comprises contacting the polypeptide aggregate fibril with a seed. The biological sample can be from an animal with an aging program perturbation. The biological sample can be from the animal without an aging program perturbation. The polypeptide aggregate fibrils include, but are not limited to, al amyloid fibrils, α-synuclein aggregates, or polyglutamine aggregates The compound includes, but is not limited to, a small chemical molecule, nucleic acid, antisense oligonucleotide, RNAi, ribozyme, oligosaccharide, antibody, polypeptide, or peptide mimetic. The compound further includes, but is not limited to, a chaperone, protease, or small heat shock protein. In one aspect, the biological disaggregation activity is denaturable. In a further aspect, the rate of fibril disappearance is measured in the presence of at least one protease inhibitor. The cellular modulator can be within an intracellular fraction or within an extracellular fraction The rate of fibril disappearance can be measured by a reduction in polypeptide aggregate fibril. In one aspect, the biological sample is from an animal with a mutation causing an aging program perturbation. The mutation causing the aging program perturbation includes, but is not limited to, daf-2, daf-16, or hsf-1 in Caenorhabditis elegans. In a further aspect, the aging program perturbation results from an RNA interference screen.

A method for identifying a cellular modulator of a biological aggregation activity of an animal is provided which comprises providing an amyloidogenic polypeptide in solution, contacting a biological sample with the amyloidogenic polypeptide, measuring a rate of fibril appearance, and identifying the modulator within the biological sample from the rate of fibril appearance in the biological aggregation activity.

In another aspect, the method further comprises measuring the rate of fibril disappearance in the presence of at least one protease inhibitor The biological disaggregation activity can be denaturable. The cellular modulator can be located within an intracellular fraction or an extracellular fraction. The polypeptide aggregate fibril includes, but is not limited to, amyloid fibrils, alpha synuclein aggregate fibrils, or polyglutamine aggregate fibrils. The rate of fibril disappearance can be measured by a reduction in amyloid fibril aggregates. The biological sample can be from an animal with a mutation causing an aging program perturbation. The mutation causing the aging program perturbation can be in daf-2, daf-16, or hsf-1 in Caenorhabditis elegans. In one aspect, the cellular modulator detoxifies polypeptide aggregate fibrils regulated by an aging program in the animal. In a further aspect, the cellular modulator detoxifies protein aggregates not regulated by an aging program in the animal.

In the methods provided herein, measuring the rate of fibril disappearance can be performed utilizing a number of techniques. In one aspect, the method further comprises measuring the rate of fibril disappearance by measuring a reduction in fluorescence of fibril-binding environment sensitive fluorophores. In a detailed aspect, the fluorophores exhibit stronger fluorescence or wavelength-shifted fluorescence, or a combination thereof, when bound to fibrils compared to when the fluorophores are solvated in aqueous medium. The fluorophore includes, but is not limited to, thioflavin T/S or Congo red. The method further comprises measuring the rate of fibril appearance by atomic force microscopy, electron microscopy or light microscopy. The method further comprises measuring the rate of fibril appearance by an increase in anisotropy of fluorescently labeled solubilized amyloidogenic peptides. The method further comprises measuring the rate of fibril appearance by an increase in turbidity or light scattering of the biological sample. The method further comprises measuring the rate of fibril appearance by disappearance of monomers or low molecular weight oligomers of amyloid fibrils. The method further comprises measuring the disappearance of monomers or low molecular weight oligomers is detected by gel electrophoresis, spectroscopically, chromomatographically, mass spectrometry or liquid chromatography mass spectrometry (LCMS). The method further comprises measuring an increase in aggregates by SDS polyacrylamide gel electrophoresis followed by Western blotting. The method further comprises measuring an increase in aggregates by sucrose gradient centrifugation. The method further comprises measuring an increase in aggregates by native gel electrophoresis visualized by antibodies or amyloidophilic dyes. The method further comprises providing an optimal quantity of seeds to the amyloidogenic polypeptide in the solution to observe the biological aggregation activity.

A method for identifying a compound which modulates biological aggregation activity in a biological sample is provided which comprises providing an amyloidogenic polypeptide or analog thereof in a solution, contacting the compound with the amyloidogenic polypeptide, providing a biological sample from an animal in an amount selected to be effective to modulate biological aggregation activity, measuring a rate of fibril appearance in the presence of the compound compared to a rate of fibril appearance in the absence of the compound, and detecting an effect of the compound on the biological aggregation activity, effectiveness of the compound being indicative of an increase in biological aggregation activity. In one aspect, the amyloid fibril is a labeled amyloid fibril prepared in vitro. The label can be a fluorophore. The label includes, but is not limited to, thioflavin T/S or Congo red. The amyloid fibril can be derived from a biological source. In one aspect, the method further comprises contacting the amyloid fibril with a seed. The biological sample can be from an animal with an aging program perturbation. The biological sample can be from the animal without an aging program perturbation. The polypeptide aggregate fibrils include, but are not limited to, aβ amyloid fibrils, α-synuclein aggregates, or polyglutamine aggregates The compound includes, but is not limited to, a small chemical molecule, nucleic acid, antisense oligonucleotide, RNAi, ribozyme, oligosaccharide, antibody, polypeptide, or peptide mimetic. The compound further includes, but is not limited to, a chaperone, protease, or small heat shock protein. In one aspect, the biological disaggregation activity is denaturable. In a further aspect, the rate of fibril disappearance is measured in the presence of at least one protease inhibitor. The cellular modulator can be within an intracellular fraction or within an extracellular fraction The rate of fibril disappearance can be measured by a reduction in polypeptide aggregate fibril. In one aspect, the biological sample is from an animal with a mutation causing an aging program perturbation. The mutation causing the aging program perturbation includes, but is not limited to, in daf-2, daf-16, or hsf-1 in Caenorhabditis elegans. In a further aspect, the aging program perturbation results from an RNA interference screen.

In the methods provided herein, measuring the rate of fibril appearance can be performed utilizing a number of techniques. In one aspect, the method further comprises measuring an increase in polypeptide aggregate fibrils. The biological sample can be from an animal with a mutation causing an aging program perturbation. The mutation causing the aging program perturbation includes, but is not limited to, a mutation in daf-2, daf-16, or hsf-1 in Caenorhabditis elegans. The aging program perturbation can result from an RNA interference screen. In a further aspect, the method comprises providing an optimal quantity of seeds to the amyloidogenic polypeptide in the solution to observe the biological aggregation activity.

### BRIEF DESCRIPTION OF THE DRAWINGS

FIGS. 1A, 1B, 1C, 1D and 1E show that attenuated IIS signaling delays aging-associated proteotoxicity of Aβ₁₋₄₂ expressed in C. elegans.

FIG. 2 shows paralysis of Aβ₁₋₄₂ C. elegans worm and increased mobility of same worm treated with daf-2 RNAi.

FIG. 3 shows minimal paralysis of wild-type worms detected through day 12 of adulthood.

FIG. 4 shows hsf-1 RNAi bacteria effectively prevents induction of HSF-1 target gene expression.

FIG. 5 shows dilutions of daf-16 and of hsf-1 RNAi do not influence their toxic effect.

FIGS. 6A and 6B show by quantitative RT-PCR and western blotting that in all RNAi treatments, mRNA levels and protein levels of Aβ₁₋₄₂ are nearly identical.

FIGS. 7A, 7B and 7C, shows schematic description of PDS preparation.

FIGS. 8A, 8B, 8C, 8D, 8E, 8F and 8G show lack of correlation between Aβ₁₋₄₂ high-MW aggregates and toxicity.

FIGS. 9A and 9B show the in-vitro kinetic aggregation assay is at least three orders more sensitive than WB.

FIGS. 10A, 10B and 10C show sonication disrupts large Aβ₁₋₄₀ fibrils into smaller fibrils.

FIG. 11 shows the lag phase shortening associated with seeding of in-vitro kinetic aggregation assay completely depends upon the presence of Aβ₁₋₄₂.

FIG. 12 shows AFM images of ultracentrifugation pellets from PDS of EV grown, hsf-1 RNAi treated worms and in-vitro aggregated Aβ₁₋₄₀

FIGS. 13A, 13B and 13C show immunoelectron microscopy of Aβ₁₋₄₂ worm samples.

FIG. 14 shows 10 μM Epoxomicin effectively inhibits proteasome activity of worm PDS.

FIGS. 15A, 15B, 15C, 15D, 15E and 15F show hsf-1 is required for efficient disaggregation of Aβ₁₋₄₂ aggregates.

FIG. 16 shows resazurin assay (Kenyon, Cell 120: 449, 2005) indicates that disaggregation activity possessed by worm homogenate reduces the toxicity of in-vitro aggregated Aβ₁₋₄₀ fibrils on PC12 cells

FIGS. 17A and 17B show intensity of an Aβ immuno-reactive 16 kDa band correlates with toxicity.

FIG. 18 shows a model of age regulated HSF-1 and DAF-16 opposing anti-proteotoxicity activities.

### DETAILED DESCRIPTION

Methods for assaying aggregation and disaggregation of macromolecules are provided to discover molecules regulated by the aging program and that ameliorate proteotoxicity and neurodegeneration associated with disease, for example, Alzheimer's disease, Parkinson's disease, Huntington's disease and related diseases. Method for identifying therapeutic compounds that are useful for treatment of disease are provided, wherein the methods utilize an assay for aggregation and disaggregation of macromolecules. Aggregation-mediated Aβ₁₋₄₂ toxicity associated with Alzheimer's Disease was reduced in C. elegans when aging was slowed by decreased insulin/insulin growth factor (IGF)-1-like signaling (IIS). The downstream transcription factors, heat shock factor-1 (HSF-1) and DAF-16 regulate opposing disaggregation and aggregation activities to promote cellular survival in response to constitutive toxic protein aggregation. Because the IIS pathway is central to the regulation of longevity and youthfulness in worms, flies and mammals, these results suggest a mechanistic link between the aging process and aggregation-mediated proteotoxicity. Methods are provided to assay and to characterize the biological disaggregation and aggregation assays that ameliorate toxicity. The methods are provided herein to discover the molecular basis of biological aggregation or active aggregation activities to detoxify intermediate MW protein aggregates. The methods are useful for discovery of therapeutic compounds for treatment of disease related to the accumulation of toxic aggregates, for example, Alzheimer's disease, Huntington's disease, Parkinson's disease, familial amyloid diseases, and related diseases.

### Biological Disaggregation Activity

A method whereby a preformed amyloid fibril is disassembled or disassembled and proteolytically degraded by a biological extract whose activity is denaturable (e.g., by heating) is provided to discover molecular biological disaggregation activities. An amyloidogenic polypeptide or analog thereof is transformed into amyloid fibrils in vitro. Intracellular or extracellular biological fractions from organisms with and without aging program perturbation are then applied to the amyloid fibrils to discern their rate of fibril disappearance in the presence and absence of protease inhibitors versus buffer controls. The presence of a protease inhibitor cocktail, optionally including inhibitors of the proteasome, allow amyloid disassembly activity to be uncoupled from proteolytic degradation of the fibrils or components thereof.

An amyloid fibril refers to a cross β-sheet aggregate having a fibril morphology or the precursors of these fibrils with minimal defined secondary or quaternary structure and lacking a clearly defined fibril morphology. Fibrils and their precursors generally bind to environment sensitive fluorophores and dyes such as Congo red and thioflavin T/S.

The molecular basis for amyloid disassembly or disassembly and proteolysis activities can be identified and quantified using intra and/or extracellular organismal fractions by the methods outlined below. Aging program perturbation utilizing RNA interference, or other inhibitors of gene expression, against one or more of the receptors, e.g., insulin/insulin like signaling receptors, the linked regulatory transcription factors, or the down stream effectors, e.g., chaperones, folding enzymes, proteases and the like or macromolecular complexes thereof, can be used with or without protease inhibitors to segregate the disaggregation and proteolysis activities that could be in the same macromolecular complexes.

The assays below can also be used to discover small molecule and macromolecular agonists and antagonists of the disassembly activities. The molecular basis for amyloid disassembly or disassembly and proteolysis activities can be discovered and quantified using activity assays including, but not limited to, the following:
1. Reduction in the fluorescence of amyloid binding environment sensitive fluorophores like thioflavin T/S or Congo red that exhibit much stronger fluorescence when bound to amyloid fibrils than solvated in aqueous medium.
2. Aggregate disappearance by atomic force microscopy, electron microscopy or by light microscopy
3. Decrease in fluorescence anisotropy of fluorescently labeled solubilized amyloidogenic peptides
4. Decrease in turbidity or light scattering of the sample
5. Appearance of monomers or low MW oligomers detected by gel electrophoresis, spectroscopically, chromomatographically or utilizing mass spectrometry or LCMS (with a protease inhibitor or inhibitor cocktail).
6. Reduction of aggregates as discerned by SDS polyacrylamide gel electrophoresis followed by Western blotting
7. Reduction of aggregates by sucrose gradient centrifugation
8. Reduction of aggregates by native gel electrophoresis visualized by antibodies or amyloidophilic dyes
9. Reduction of toxic aggregates can be followed using cell-line based toxicity assays or primary neurons
10. Reduction of aggregates as discerned by separations based on aggregation state including centrifugation and ultracentrifugation, gel filtration, membrane filtration or an analogous method.

The aggregation activities as outlined below will also be inhibited with RNAi or small chemical molecules which should hasten the disaggregation activities.

These biological disaggregation activities may have generic and disease specific aspects. Therefore we employ a variety of organismal models of Alzheimer's disease including, but not limited to, C. elegans, D. melanogaster, mice, and in human patient samples to discover activities that could take apart Aβ aggregates including fibrils inside and/or outside the cell to ameliorate Alzheimer's disease. A key question in these diseases is whether intracellular or extracellular aggregates mediate the proteotoxicity, a question that can be answered by utilizing both intra- and extracellular proteotoxicity models in combination with aging program perturbation using RNAi and/or small molecule perturbation.

These biological disaggregation activities will be sought in a variety of organismal models of Parkinson's disease, including, but not limited to, C. elegans, D. melanogaster, mice, and in human patient samples will also be used to discover activities that could take apart alpha synuclein aggregates or Lewy Bodies including fibrils inside and/or outside the cell to ameliorate Parkinson's disease.

These biological disaggregation activities will be sought in a variety of organismal models of Huntington's disease, including, but not limited to, C. elegans, D. melanogaster, mice, and in human patient samples to discover activities that could take apart polyglutamine aggregates including fibrils inside and/or outside the cell to ameliorate Huntington's disease.

These biological disaggregation activities will be used in a variety of organismal models of familial amyloid diseases including amyloid diseases involving gelson, transthyretin, and immunoglobulin light chain misfolding or misfolding and deposition, including, but not limited to, C. elegans, D. melanogaster, and mice. Amyloid disease involving immunoglobulin light chain include, but are not limited to, immunoglobulin light chain amyloidosis; Amyloid disease involving trans-thyretin, include, but are not limited to, familial amyloid polyneuropathy; familial amyloid cardiomyopathy or senile systemic amyloidosis. Human patient samples will also be utilized to discover activities that could take apart protein aggregates including fibrils inside and/or outside the cell to ameliorate these peripheral or CNS diseases. Again, a key question is whether intra- or extracellular aggregates mediate these diseases. The methods of the present invention provides a means to identify both intra- and extracellular activities which can be inhibited to discern the intra- or extra-cellular origins of proteotoxicity.

### Aggregation Assay to Detect Biological Aggregation

A method is provided to identify the molecular basis of biological aggregation or active aggregation activities to detoxify intermediate MW protein aggregates. These activities are identified by transforming seed or nucleus free mostly or completely monomeric amyloidogenic polypeptide or analog thereof in a sub μM to μM range into amyloid fibrils by a biological activity that is denaturable (e.g. by heat or chaotrope denaturation—both enabling fibril formation). Heat inactivation must be carefully monitored to avoid producing heat induced fibrils that can serve as seeds.

An amyloidogenic peptide or protein is rendered aggregate or seed free by a process such as gel filtration or membrane filtration. Intra and extracellular biological fractions from organisms with and without aging program perturbation and/or with and without inhibitors of disaggregation activities described above are added to a seed free amyloidogenic polypeptide to discern their rate of fibril formation in the presence and absence of protease inhibitors versus buffer controls. RNAi or small molecule antagonists against the disaggregation activities described above are envisioned to be very useful to uncouple the active aggregation and disaggregation activities discussed above. The presence of a protease inhibitor cocktail, optionally including inhibitors of the proteasome, allow active amyloid aggregation activity to be uncoupled from proteolytic degradation of the fibrils or components thereof.

The presence of seeds will hasten the rate of amyloidogenesis according to the principles of a nucleated polymerization, hence the background rate enhancement from seeding by preformed aggregates in the organismal extract can be differentiated from that caused by an active aggregation process by comparing aggregation rates with and without the active aggregation and disaggregation activities inhibited. This can be achieved by the following means:

Heat denaturation occurs at a temperature that knocks out active aggregation activity, but does not disaggregate Aβ fibrils. These experiments are conducted in the presence and absence of disaggregation activity (i.e., ±inhibitors).

Aβ₁₋₄₂ worms are compared to worms not expressing Aβ₁₋₄₂ in the presence and absence of disaggregation activity (±inhibitors) in order to discern active aggregation inhibition with seeds to active aggregation without seeds.

The denatured organismal extracts (using methods such as heat inactivation, pH manipulations or the addition of additives that denature such as urea) will be compared with non-denatured extracts which will accelerate amyloidogenic polypeptide amyloid formation due to seeding and active aggregation. Hence the rate will be faster than the denatured biological sample which only has activity derived from the seeds.

RNAi inhibition of candidate active aggregation genes are used to discern active aggregation rate differences. These experiments will be carried out in the presence and absence of disaggregation activity (i.e., ±inhibitors).

Exogenous amyloidogenic peptide having a distinct sequence that will not cross-seed with Aβ but which is a substrate for active aggregation will be used to read out active aggregation. These experiments will be carried out in the presence and absence of disaggregation activity inhibitors.

A variation on this assay includes an iteration wherein a known quantity of seeds are added to a nucleus free amyloidogenic polypeptide or analog thereof that is transformed into amyloid fibrils by a biological aggregation activity that is denaturable. Some active aggregation activities may require a preformed aggregate to achieve active aggregation, an aggregate or seed that is not in the organismal extract due to an aging program perturbation. These experiments will be carried out with and without inhibition of the active disaggregation activity.

Active aggregation kinetics as a function of the conditions described above can be followed by:
1. Increase in the fluorescence of amyloid binding environment sensitive fluorophores like thioflavin T/S or Congo red that exhibit much stronger fluorescence when bound to amyloid fibrils than when solvated in aqueous medium.
2. Appearance of aggregates or fibrils by atomic force microscopy or electron microscopy or by light microscopy
3. Increase in fluorescence anisotropy of fluorescently labeled amyloidogenic peptides
4. Increase in turbidity or light scattering of the sample
5. Disappearance of monomers or low MW oligomers detected by gel electrophoresis, spectroscopically, chromomatographically or utilizing mass spectrometry
6. Increased aggregates by SDS Page followed by western blotting
7. Increased aggregates by Sucrose gradient centrifugation
8. Increased aggregates by Native gel electrophoresis visualized by antibodies or amyloidophilic dyes
9. Readout of a biological function such as toxicity changes in a cell line or primary neurons
10. Increase in high MW fraction as discerned by separations based on aggregation state including centrifugation and ultracentrifugation, gel filtration, membrane filtration or an analogous filtration method.

The disaggregation activities outlined above will be analyzed by inhibition by small molecule antagonists, RNAi, or other inhibitors, which is expected to enhance the kinetics of the active aggregation activity described.

These activities will be identified and characterized in a variety of organismal models of Alzheimer's disease including, but not limited to, C. elegans, D. melanogaster, and mice. Analogous activities will also be sought in human samples to discover activities that could detoxify small or low MW Aβ aggregates including fibrils inside and/or outside the cell by making high MW aggregates to ameliorate Alzheimer's disease. A key question that may be resolvable by using intra and extracellular animal extracts is to discern whether intra or extracellular aggregation causes Alzheimer's disease.

These activities will be identified and characterized in a variety of organismal models of Parkinson's disease, including, but not limited to, C. elegans, D. melanogaster, and mice. Human patient samples will also be used to discover activities that could actively aggregate alpha synuclein aggregates including low MW aggregates inside and/or outside the cell to ameliorate Parkinson's disease.

These activities will be identified and characterized in a variety of organismal models of Huntington's disease, including, but not limited to, C. elegans, D. melanogaster, and mice. Human patient samples will also be studied to discover activities that could actively aggregate polyglutamine aggregates including low MW aggregates inside and/or outside the cell to ameliorate Huntington's disease.

These biological aggregation activities will be identified and characterized in a variety of organismal models of familial amyloid diseases including amyloid diseases involving the misfolding or misfolding and aggregation of gelson, transthyretin, and light chain proteins. Organisms include, but are not limited to, C. elegans, D. melanogaster, and mice. Human patient samples will also be utilized to discover activities that can actively aggregate low MW aggregates inside and/or outside the cell to ameliorate these peripheral or CNS diseases. Again, a key question is whether intra- or extracellular aggregates mediate these diseases. The methods provided herein provide a means to discover both intra- and extracellular activities which can be inhibited to discern the intra or extracellular origins of proteotoxicity.

Methods for identifying a cellular modulator of a biological disaggregation activity or a biological aggregation activity in an animal will be used to identify the constitutive extracellular or intracellular molecular mediators of active aggregation and disaggregation to detoxify protein aggregation not regulated by the aging program

The methods will be used to identify the extracellular or intracellular molecular mediators of active aggregation and disaggregation that detoxify protein aggregation regulated by the aging program

The methods will be used to identify small molecule (<1000 MW) modulators of active aggregation and disaggregation activities both inside and outside of the cell not associated with the aging program.

The methods will be used to identify small molecule (<1000 MW) modulators of active aggregation and disaggregation activities both inside and outside of the cell regulated by the aging program

The methods will be used to identify macromolecular (>1000 MW) modulators of active aggregation and disaggregation activities both inside and outside of the cell not associated with the aging program using RNA interference screens and the equivalent.

The methods will be used to identify macromolecular (>1000 MW) modulators of active aggregation and disaggregation activities both inside and outside of the cell, activities that are regulated by the aging program using RNA interference screens and the equivalent.

Macromolecules include, but are not limited to, proteins, oligosaccharides, nucleic acids.

Macromolecules include, but are not limited to, chaperones, proteases, related proteins and combinations thereof. Macromolecules include, but are not limited to, small heat shock proteins. Macromolecules can be small heat shock proteins that make quaternary interactions with other macromolecules that mediate active aggregation or disaggregation or disaggregation coupled to proteolysis.

The methods will be used to identify the aggregate or distribution of aggregates that lead to proteotoxicity both inside and outside of the cell. The biological activities described above should identify their substrates and thus the toxic species.

The methods are used to identify the extracellular or intracellular molecular mediators of active aggregation and disaggregation may also reveal the mechanism of proteotoxicity either directly through proteomics or indirectly utilizing genetics and signaling pathway information.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Biological disaggregation activity” refers to an activity exhibited by an organism that disassembles or disassembles and proteolyzes toxic aggregates including amyloid

“Biological aggregation activity” refers to an activity exhibited by an organism that assembles or aggregates toxic aggregates into less toxic higher MW aggregates

“Amyloidogenic polypeptide” refers to a polypeptide that has the propensity to aggregate into amyloid fibrils that may or may not be associated with a neurodegenerative disease

“Seed” or “seeding” refers to a nucleated polymerization requires the formation of an oligomeric high energy species before the aggregation or amyloid fibril reaction becomes spontaneous. A seed is an aggregate larger than the nucleus the obviates the need for nucleation and renders the aggregation reaction spontaneous from the outset.

“Polypeptide aggregate fibril” refers to macromolecules made of 20 natural amino acids connected by amide bonds. Polypeptides, peptides and proteins are largely synonymous. Sources of polypeptide aggregate fibrils include, but are not limited to, neuronal cells, brain tissue, animal or bacterial cells engineered to express polypeptide aggregate fibrils; organisms, e.g., mammals or non-mammals, Cenorhabditis elegans, or Drosophila melanogaster, engineered to express polypeptide aggregate fibrils.

“Optimal quantity of seeds” refers to the amount of seeds to accelerate aggregate fibril formation. The influence of seeding is usually experimentally observable in accelerating the rate of fibril formation when the seeds are added to a seed free reaction at a concentration above 1% by weight. The rate acceleration also depends of the shear rate of the growing fibrils and the sheer rate can be increased by agitation

“Patient”, “subject”, “vertebrate” or “mammal” are used interchangeably and refer to mammals such as human patients and non-human primates, as well as experimental animals such as rabbits, rats, and mice, and other animals. Animals include all vertebrates and invertebrates, e.g., mammals and non-mammals, such as sheep, dogs, cows, chickens, Cenorhabditis elegans, Drosophila melanogaster, amphibians, and reptiles.

“Treating” or “treatment” includes the administration of the small chemical molecule, antibody, shRNA or RNAi compositions, compounds or agents of the present invention to prevent or delay the onset of the symptoms, complications, or biochemical indicia of a disease, alleviating the symptoms or arresting or inhibiting further development of the disease, condition, or disorder, such as disease related to the accumulation of toxic aggregates, for example, Alzheimer's disease, Huntington's disease, and Parkinson's disease. Treatment can be prophylactic (to prevent or delay the onset of the disease, or to prevent the manifestation of clinical or subclinical symptoms thereof) or therapeutic suppression or alleviation of symptoms after the manifestation of the disease.

“Inhibitors,” “activators,” and “modulators” of disaggregation activity or aggregation activity in cells are used to refer to inhibitory, activating, or modulating molecules, respectively, identified using in vitro and in vivo assays for disaggregation activity or aggregation activity, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate disaggregation activity or aggregation activity, e.g., antagonists. Activators are agents that, e.g., bind to, stimulate, increase, open, activate, facilitate, enhance activation, sensitize or up regulate disaggregation activity or aggregation activity, e.g., agonists. Modulators include agents that, e.g., alter the interaction of amyloidogenic polypeptides with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of biological molecules with disaggregation activity or aggregation activity, e.g., with altered activity, as well as naturally occurring and synthetic ligands, antagonists, agonists, small chemical molecules and the like. “Cell-based assays” for inhibitors and activators include, e.g., applying putative modulator compounds to a biological sample having disaggregation activity or aggregation activity and then determining the functional effects on disaggregation activity or aggregation activity, as described herein. “Cell based assays” include, but are not limited to, in vivo tissue or cell samples from a mammalian subject or in vitro cell-based assays comprising a biological sample having disaggregation activity or aggregation activity that are treated with a potential activator, inhibitor, or modulator are compared to control samples without the inhibitor, activator, or modulator to examine the extent of inhibition. Control samples (untreated with inhibitors) can be assigned a relative disaggregation activity or aggregation activity value of 100%. Inhibition of disaggregation activity or aggregation activity is achieved when the disaggregation activity or aggregation activity value relative to the control is about 80%, optionally 50% or 25-0%. Activation of disaggregation activity or aggregation activity is achieved when the disaggregation activity or aggregation activity value relative to the control is 110%, optionally 150%, optionally 200-500%, or 1000-3000% higher.

A method for identifying a cellular modulator of a biological disaggregation activity in an animal is provided which comprises contacting an amyloid fibril or precursor thereof to disassemble it, contacting a biological sample with the polypeptide aggregate fibrils, measuring a rate of fibril disappearance in the presence of protease inhibitors, and identifying the modulator within the biological sample from the rate of fibril disappearance. A method for identifying a compound which modulates biological disaggregation activity in a biological sample is provided.

A method for identifying a cellular modulator of a biological aggregation activity in an animal is provided which comprises providing a seed-free or nucleus-free amyloidogenic polypeptide, contacting a biological sample with the amyloidogenic polypeptide, measuring a rate of fibril appearance in the presence at least one protease inhibitor, and identifying the modulator within the biological sample from the rate of fibril appearance. A method for identifying a compound which modulates biological aggregation activity in a biological sample is provided.

“Compound” or “test compound” refers to any compound tested as a modulator of disaggregation activity or aggregation activity. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators of biological activities that affect a disaggregation activity or aggregation activity. Typically, test compounds will be small organic molecules, peptides, lipids, or lipid analogs.

In one embodiment, antibody binding to a cellular receptor signaling disaggregation activity or aggregation activity, e.g., insulin/IGF-1 receptor, can be assayed by either immobilizing the ligand or the receptor. For example, the assay can include immobilizing cellular receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Signaling responsiveness” refers to signaling via the IIS pathway, for example, in mice or in the nematode Caenorhabditis elegans signaling via the insulin/IGF-1 receptor, DAF-2, initiates the transduction of a signal that causes the phosphorylation of the FOXO transcription factor, DAF-16. Recent studies indicate that in worms, life-span extension due to reduced daf-2 activity is also dependent upon the Heat Shock Factor 1 (HSF-1). Moreover, increased expression of hsf-1 extends worm lifespan in a daf-16 dependent manner. Signal generating compounds for measurement in cell-based assays can be generated, e.g., by conjugation with an enzyme or fluorophore, e.g., thioflavin T/S or Congo red. Enzymes of interest as labels will primarily be hydrolases, particularly kinases, phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

“Detecting an effect of a test compound on disaggregation activity or aggregation activity” can refer to a therapeutic or prophylactic effect in a mammalian subject, such as the reduction, elimination, or prevention of the disease, symptoms of the disease, or side effects of the disease in the subject. “Detecting an effect of a test compound on disaggregation activity or aggregation activity” can refer to a compound having an effect in a cell-based assay, e.g., a diagnostic assay, as measured by IGF-1 signaling via hsf-1 or daf-16, for example, in C. elegans. A loss-of-function mutation in the hsf-1, daf-2 or daf-16 genes, can affect disaggregation activity or aggregation activity, and the onset or prognosis for protein aggregation diseases, for example, Alzheimer's disease, Huntington's disease, Parkinson's disease, or familial amyloid disease.

### High Throughput Assays for Modulators of Disaggregation Activity or Aggregation Activity

The compounds tested as modulators of disaggregation activity or aggregation activity can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of a cellular modulator of disaggregation activity or aggregation activity. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, R U, 3D Pharmaceuticals, Exton, Pa., Martek Biosciences, Columbia, Md., etc.).

Candidate compounds are useful as part of a strategy to identify drugs for treating disorders involving aggregation and disaggregation of macromolecules wherein the compounds modulate activity of cellular molecules regulated by the aging program. The compounds ameliorate proteotoxicity and neurodegeneration associated with Alzheimer's disease, Parkinson's disease, Huntington's disease and related diseases. A test compound that binds to a cellular modulator of disaggregation activity or aggregation activity is considered a candidate compound.

Screening assays for identifying candidate or test compounds that bind to one or more cellular modulators of disaggregation activity or aggregation activity, or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are dominant negative variants of cellular modulators of disaggregation activity or aggregation activity.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of a cellular modulator of disaggregation activity or aggregation activity, or a biologically active portion thereof can be determined, e.g., by monitoring disaggregation activity or aggregation activity as measured by disappearance or appearance of polypeptide aggregate fibrils in the presence of the test compound. The ability of the test compound to modulate disaggregation activity or aggregation activity can also be determined by wherein the cellular modulator is an intracellular molecule or an extracellular molecule. The binding assays can be cell-based or cell-free.

Methods are provided for assaying aggregation and disaggregation of macromolecules to discover molecules regulated by the aging program and that ameliorate proteotoxicity and neurodegeneration associated with Alzheimer's disease, Parkinson's disease, Huntington's disease and related diseases. These can be determined by one of the methods described herein or known in the art for determining direct binding. In one embodiment, Candidate compounds are useful as part of a strategy to identify drugs for treating disorders involving aggregation and disaggregation of macromolecules wherein the compounds modulate activity of cellular molecules regulated by the aging program. The compounds ameliorate proteotoxicity and neurodegeneration associated with Alzheimer's disease, Parkinson's disease, Huntington's disease and related diseases. A test compound that binds to a cellular modulator of disaggregation activity or aggregation activity is considered a candidate compound. Detection of a cellular modulator of disaggregation activity or aggregation activity can be determined by methods known in the art including, but not limited to, measuring the rate of fibril appearance or disappearance by an increase or reduction, respectively, in the fluorescence of amyloid binding fluorophores, for example, thioflavin T/S or Congo red; measuring the rate of fibril appearance or disappearance by atomic force microscopy, electron microscopy or light microscopy; measuring the rate of fibril appearance or disappearance by an increase in anisotropy of fluorescently labeled solubilized amyloidogenic peptides; measuring the rate of fibril appearance or disappearance by a decrease in turbidity or light scattering of the biological sample; measuring the rate of fibril appearance or disappearance by appearance of monomers or low molecular weight oligomers of amyloid fibrils; measuring the appearance of monomers or low molecular weight oligomers is detected by gel electrophoresis, spectroscopically, chromomatographically, mass spectrometry or LCMS; measuring a increase or reduction of aggregates by SDS polyacrylamide gel electrophoresis followed by Western blotting; measuring a increase or reduction of aggregates by sucrose gradient centrifugation; or measuring a reduction of aggregates by native gel electrophoresis visualized by antibodies or amyloidophilic dyes.

This invention further pertains to novel agents identified by the above-described screening assays and uses thereof for treatments as described herein.

In one embodiment the invention provides soluble assays using a cellular modulator of disaggregation activity or aggregation activity, or a cell or tissue expressing a cellular modulator of disaggregation activity or aggregation activity, either naturally occurring or recombinant. In another embodiment, the invention provides solid phase based in vitro assays in a high throughput format, where a cellular modulator of disaggregation activity or aggregation activity is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for a cellular modulator of disaggregation activity or aggregation activity in vitro, or for cell-based or membrane-based assays comprising a cellular modulator of disaggregation activity or aggregation activity. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin). Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Inhibiting Expression of Polypeptides and Transcripts

The invention further provides for nucleic acids complementary to (e.g., antisense sequences to) cellular modulators of disaggregation activity or aggregation activity. Antisense sequences are capable of inhibiting the transport, splicing or transcription of protein-encoding genes, e.g., nucleic acids encoding daf-2, daf-16, or hsf-1 in Caenorhabditis elegans. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

Antisense Oligonucleotides. The invention provides antisense oligonucleotides capable of binding messenger RNA, e.g., mRNA encoding daf-2, daf-16, or hsf-1 in Caenorhabditis elegans which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

siRNA. “Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. Preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

Inhibitory Ribozymes. The invention provides ribozymes capable of binding message which can inhibit polypeptide activity by targeting mRNA, e.g., inhibition of polypeptides with daf-2, daf-16, or hsf-1 activity, e.g., disaggregation activity or aggregation activity. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

### Antibodies as Modulators of Disaggregation Activity or Aggregation Activity

The antibodies and antigen-binding fragments thereof described herein specifically bind to a cellular modulator to affect disaggregation activity or aggregation activity.

In some embodiments, the antibody or antigen-binding fragment thereof or selectively binds (e.g., competitively binds, or binds to same epitope, e.g., a conformational or a linear epitope) to an antigen that is selectively bound by an antibody produced by a hybridoma cell line. Thus, the epitope can be in close proximity spatially or functionally-associated, e.g., an overlapping or adjacent epitope in linear sequence or conformational space, to a known epitope bound by an antibody. Potential epitopes can be identified computationally using a peptide threading program, and verified using methods known in the art, e.g., by assaying binding of the antibody to a cellular modulator of disaggregation activity or aggregation activity.

Methods of determining the sequence of an antibody described herein are known in the art; for example, the sequence of the antibody can be determined by using known techniques to isolate and identify a cDNA encoding the antibody from the hybridoma cell line. Methods for determining the sequence of a cDNA are known in the art.

The antibodies described herein typically have at least one or two heavy chain variable regions (V_{H}), and at least one or two light chain variable regions (V_{L}). The V_{H} and V_{L} regions can be further subdivided into regions of hypervariability, termed complementarity determining regions (CDR), which are interspersed with more highly conserved framework regions (FR). These regions have been precisely defined (see, Kabat et al., Sequences of Proteins of Immunological Interest, Fifth Edition, U.S. Department of Health and Human Services, NIH Publication No. 91-3242, 1991 and Chothia et al., J. Mol. Biol. 196: 901-917, 1987). Antibodies or antibody fragments containing one or more framework regions are also useful in the invention. Such fragments have the ability to specifically bind to a cellular modulator of disaggregation activity or aggregation activity.

An antibody as described herein can include a heavy and/or light chain constant region (constant regions typically mediate binding between the antibody and host tissues or factors, including effector cells of the immune system and the first component (C1q) of the classical complement system), and can therefore form heavy and light immunoglobulin chains, respectively. For example, the antibody can be a tetramer (two heavy and two light immunoglobulin chains, which can be connected by, for example, disulfide bonds). The antibody can contain only a portion of a heavy chain constant region (e.g., one of the three domains heavy chain domains termed C_{H}1, C_{H}2, and C_{H}3, or a portion of the light chain constant region (e.g., a portion of the region termed C_{L}).

Antigen-binding fragments are also included in the invention. Such fragments can be: (i) a F_{ab} fragment (i.e., a monovalent fragment consisting of the V_{L}, V_{H}, C_{L}, and C_{H}1 domains); (ii) a F(_{ab}′)₂ fragment (i.e., a bivalent fragment containing two F_{ab} fragments linked by a disulfide bond at the hinge region); (iii) a F_{d} fragment consisting of the V_{H} and C_{H}1 domains; (iv) a Fᵥ fragment consisting of the V_{L} and V_{H} domains of a single arm of an antibody, (v) a dAb fragment (Ward et al., Nature 341: 544-546, 1989), which consists of a V_{H} domain; and/or (vi) an isolated complementarity determining region (CDR).

Fragments of antibodies (including antigen-binding fragments as described above) can be synthesized using methods known in the art such as in an automated peptide synthesizer, or by expression of a full-length gene or of gene fragments in, for example, E. coli. F(_{ab}′)₂ fragments can be produced by pepsin digestion of an antibody molecule, and F_{ab} fragments can be generated by reducing the disulfide bridges of F(_{ab}′)₂ fragments. Alternatively, F_{ab} expression libraries can be constructed (Huse et al., Science 246: 1275-81, 1989) to allow relatively rapid identification of monoclonal F_{ab} fragments with the desired specificity.

Methods of making other antibodies and antibody fragments are known in the art. For example, although the two domains of the Fv fragment, V_{L} and V_{H}, are coded for by separate genes, they can be joined, using recombinant methods or a synthetic linker that enables them to be made as a single protein chain in which the V_{L} and V_{H} regions pair to form monovalent molecules (known as single chain Fv (scFv); see e.g., Bird et al., Science 242: 423-426, 1988; Huston et al., Proc. Natl. Acad. Sci. USA 85: 5879-5883, 1988; Colcher et al., Ann. NY Acad. Sci. 880: 263-80, 1999; and Reiter, Clin. Cancer Res. 2: 245-52, 1996).

Techniques for producing single chain antibodies are also described in U.S. Pat. Nos. 4,946,778 and 4,704,692. Such single chain antibodies are encompassed within the term “antigen-binding fragment” of an antibody. These antibody fragments are obtained using conventional techniques known to those of ordinary skill in the art, and the fragments are screened for utility in the same manner that intact antibodies are screened. Moreover, a single chain antibody can form complexes or multimers and, thereby, become a multivalent antibody having specificities for different epitopes of the same target protein.

Antibodies and portions thereof that are described herein can be monoclonal antibodies, generated from monoclonal antibodies, or can be produced by synthetic methods known in the art. Antibodies can be recombinantly produced (e.g., produced by phage display or by combinatorial methods, as described in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9: 1370-1372, 1991; Hay et al., Human Antibody Hybridomas 3: 81-85, 1992; Huse et al., Science 246: 1275-1281, 1989; Griffiths et al., EMBO J. 12: 725-734, 1993; Hawkins et al., J. Mol. Biol. 226: 889-896, 1992; Clackson et al., Nature 352: 624-628, 1991; Gram et al., Proc. Natl. Acad. Sci. USA 89: 3576-3580, 1992; Garrad et al., Bio/Technology 9: 1373-1377, 1991; Hoogenboom et al., Nucl. Acids Res. 19: 4133-4137, 1991; and Barbas et al., Proc. Natl. Acad. Sci. USA 88: 7978-7982, 1991).

As one example, antibody to a cellular modulator of disaggregation activity or aggregation activity can be made by immunizing an animal with a polypeptide, or fragment (e.g., an antigenic peptide fragment derived from (i.e., having the sequence of a portion of) a modulator isolated from a cellular extract of an animal, e.g., daf-2, daf-16, or hsf-1 in Caenorhabditis elegans. In some embodiments, antibodies or antigen-binding fragments thereof described herein can bind to a purified Daf-2, Daf-16 or Hsf-1. In some embodiments, the antibodies or antigen-binding fragments thereof can bind to Daf-2, Daf-16 or Hsf-1 in a tissue section, a whole cell (living, lysed, or fractionated), or a membrane fraction. Antibodies can be tested, e.g., in in vitro systems) for the ability to activate or inhibit disaggregation activity or aggregation activity via a cellular modulators in a cell extract.

In the event an antigenic peptide derived from a cellular modulator of disaggregation activity or aggregation activity is used, it will typically include at least eight (e.g., 10, 15, 20, 30, 50, 100 or more) consecutive amino acid residues of a domain of the protein. In some embodiments, the antigenic peptide will comprise all of the domain of the protein. The antibodies generated can specifically bind to one of the proteins in their native form (thus, antibodies with linear or conformational epitopes are within the invention), in a denatured or otherwise non-native form, or both. Peptides likely to be antigenic can be identified by methods known in the art, e.g., by computer-based antigenicity-predicting algorithms. Conformational epitopes can sometimes be identified by identifying antibodies that bind to a protein in its native form, but not in a denatured form.

The host animal (e.g., a rabbit, mouse, guinea pig, or rat) can be immunized with the antigen, optionally linked to a carrier (i.e., a substance that stabilizes or otherwise improves the immunogenicity of an associated molecule), and optionally administered with an adjuvant (see, e.g., Ausubel et al., supra). An exemplary carrier is keyhole limpet hemocyanin (KLH) and exemplary adjuvants, which will typically be selected in view of the host animal's species, include Freund's adjuvant (complete or incomplete), adjuvant mineral gels (e.g., aluminum hydroxide), surface active substances such as lysolecithin, pluronic polyols, polyanions, peptides, oil emulsions, dinitrophenol, BCG (bacille Calmette-Guerin), and Corynebacterium parvum. KLH is also sometimes referred to as an adjuvant. The antibodies generated in the host can be purified by, for example, affinity chromatography methods in which the polypeptide antigen or a fragment thereof, is immobilized on a resin.

Epitopes encompassed by an antigenic peptide will typically be located on the surface of the protein (e.g., in hydrophilic regions), or in regions that are highly antigenic (such regions can be selected, initially, by virtue of containing many charged residues). An Emini surface probability analysis of human protein sequences can be used to indicate the regions that have a particularly high probability of being localized to the surface of the protein.

The antibody can be a fully human antibody (e.g., an antibody made in a mouse or other mammal that has been genetically engineered to produce an antibody from a human immunoglobulin sequence, such as that of a human immunoglobulin gene (the kappa, lambda, alpha (IgA₁ and IgA₂), gamma (IgG₁, IgG₂, IgG₃, IgG₄), delta, epsilon and mu constant region genes or the myriad immunoglobulin variable region genes). Alternatively, the antibody can be a non-human antibody (e.g., a rodent (e.g., a mouse or rat), goat, rabbit, or non-human primate (e.g., monkey) antibody).

Human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368: 856-859, 1994; Green et al., Nature Genet. 7: 13-21, 1994; Morrison et al., Proc. Natl. Acad. Sci. USA 81: 6851-6855, 1994; Bruggeman et al, Immunol. 7: 33-40, 1993; Tuaillon et al., Proc. Natl. Acad. Sci. USA 90: 3720-3724, 1993; and Bruggeman et al., Eur. J. Immunol. 21: 1323-1326, 1991).

The antibody to a cellular modulator of disaggregation activity or aggregation activity can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (in, e.g., the variable framework or constant region) to decrease antigenicity in a human. Chimeric antibodies (i.e., antibodies in which different portions are derived from different animal species (e.g., the variable region of a murine mAb and the constant region of a human immunoglobulin) can be produced by recombinant techniques known in the art. For example, a gene encoding the F_{c} constant region of a murine (or other species) monoclonal antibody molecule can be digested with restriction enzymes to remove the region encoding the murine F_{c}, and the equivalent portion of a gene encoding a human F_{c} constant region can be substituted therefore (see, e.g., European Patent Application Nos. 125,023; 184,187; 171,496; and 173,494; see also WO 86/01533; U.S. Pat. No. 4,816,567; Better et al., Science 240: 1041-1043, 1988; Liu et al., Proc. Natl. Acad. Sci. USA 84: 3439-3443, 1987; Liu et al., J. Immunol. 139: 3521-3526, 1987; Sun et al., Proc. Natl. Acad. Sci. USA 84: 214-218, 1987; Nishimura et al., Cancer Res. 47: 999-1005, 1987; Wood et al., Nature 314: 446-449, 1985; Shaw et al., J. Natl. Cancer Inst. 80: 1553-1559, 1988; Morrison et al., Proc. Natl. Acad. Sci. USA 81: 6851, 1984; Neuberger et al., Nature 312: 604, 1984; and Takeda et al., Nature 314: 452, 1984).

In a humanized or CDR-grafted antibody, at least one or two, but generally all three of the recipient CDRs (of heavy and or light immunoglobulin chains) will be replaced with a donor CDR (see, e.g., U.S. Pat. No. 5,225,539; Jones et al., Nature 321: 552-525, 1986; Verhoeyan et al., Science 239: 1534, 1988; and Beidler et al., J. Immunol. 141: 4053-4060, 1988). One need replace only the number of CDRs required for binding of the humanized antibody to a cellular modulator of disaggregation activity or aggregation activity. The donor can be a rodent antibody, and the recipient can be a human framework or a human consensus framework. Typically, the immunoglobulin providing the CDRs is called the “donor” (and is often that of a rodent) and the immunoglobulin providing the framework is called the “acceptor.” The acceptor framework can be a naturally occurring (e.g., a human) framework, a consensus framework or sequence, or a sequence that is at least 85% (e.g., 90%, 95%, 99%) identical thereto. A “consensus sequence” is one formed from the most frequently occurring amino acids (or nucleotides) in a family of related sequences (see, e.g., Winnaker, From Genes to Clones, Verlagsgesellschaft, Weinheim, Germany, 1987). Each position in the consensus sequence is occupied by the amino acid residue that occurs most frequently at that position in the family (where two occur equally frequently, either can be included). A “consensus framework” refers to the framework region in the consensus immunoglobulin sequence. Humanized antibodies to a cellular modulator of disaggregation activity or aggregation activity can be made in which specific amino acid residues have been substituted, deleted or added (in, e.g., in the framework region to improve antigen binding). For example, a humanized antibody will have framework residues identical to those of the donor or to amino acid a receptor other than those of the recipient framework residue. To generate such antibodies, a selected, small number of acceptor framework residues of the humanized immunoglobulin chain are replaced by the corresponding donor amino acids. The substitutions can occur adjacent to the CDR or in regions that interact with a CDR (U.S. Pat. No. 5,585,089, see especially columns 12-16). Other techniques for humanizing antibodies are described in EP 519596 A1.

An antibody to a cellular modulator of disaggregation activity or aggregation activity can be humanized as described above or using other methods known in the art. For example, humanized antibodies can be generated by replacing sequences of the Fv variable region that are not directly involved in antigen binding with equivalent sequences from human Fv variable regions. General methods for generating humanized antibodies are provided by Morrison, Science 229: 1202-1207, 1985; Oi et al., BioTechniques 4: 214, 1986, and Queen et al. (U.S. Pat. Nos. 5,585,089; 5,693,761, and 5,693,762). The nucleic acid sequences required by these methods can be obtained from a hybridoma producing an antibody against a cellular modulator or fragments thereof having the desired properties such as the ability to activate or inhibit a disaggregation activity or an aggregation activity. The recombinant DNA encoding the humanized antibody, or fragment thereof, can then be cloned into an appropriate expression vector.

In certain embodiments, the antibody has an effector function and can fix complement, while in others it can neither recruit effector cells nor fix complement. The antibody can also have little or no ability to bind an Fc receptor. For example, it can be an isotype or subtype, or a fragment or other mutant that cannot bind to an Fc receptor (e.g., the antibody can have a mutant (e.g., a deleted) Fc receptor binding region). Antibodies lacking the Fc region typically cannot fix complement, and thus are less likely to cause the death of the cells they bind to.

In other embodiments, the antibody can be coupled to a heterologous substance, such as a therapeutic agent (e.g., an antibiotic), or a detectable label. A detectable label can include an enzyme (e.g., horseradish peroxidase, alkaline phosphatase, .beta.-galactosidase, or acetylcholinesterase), a prosthetic group (e.g., streptavidin/biotin and avidin/biotin), or a fluorescent, luminescent, bioluminescent, or radioactive material (e.g., umbelliferone, fluorescein, fluorescein isothiocyanate, rhodamine, dichlorotriazinylamine fluorescein, dansyl chloride or phycoerythrin (which are fluorescent), luminol (which is luminescent), luciferase, luciferin, and aequorin (which are bioluminescent), and ⁹⁹mTc, ¹⁸⁸Re, ¹¹¹In, ¹²⁵I, ¹³¹I, ³⁵S or ³H (which are radioactive)).

The antibodies described herein (e.g., monoclonal antibodies) can also be used to isolate cellular modulators of disaggregation activity or aggregation activity, or fragments thereof such as the fragment associated with activation or inhibition of disaggregation activity or aggregation activity, intracellularly or extracellularly (by, for example, affinity chromatography or immunoprecipitation) or to detect them in, for example, a cell lysate or supernatant (by Western blotting, enzyme-linked immunosorbant assays (ELISAs), radioimmune assays, and the like) or a histological section. These methods permit the determination of the abundance and pattern of expression of a particular protein. This information can be useful in making a diagnosis or in evaluating the efficacy of a clinical test or treatment.

The invention also includes the nucleic acids that encode the antibodies described above and vectors and cells (e.g., mammalian cells such as CHO cells or lymphatic cells) that contain them (e.g., cells transformed with a nucleic acid that encodes an antibody that specifically binds to a cellular modulator of disaggregation activity or aggregation activity). Similarly, the invention includes cell lines (e.g., hybridomas) that make the antibodies of the invention and methods of making those cell lines.

### Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., thioflavin T/S, Congo red, fluorescein isothiocyanate, Texas red, or rhodamine), radiolabels (e.g., ³H, ¹²⁵I, ³⁵S, ¹⁴C, or ³²P), enzymes (e.g., horse radish peroxidase, alkaline phosphatase and others commonly used in an ELISA), chemiluminescent labels, and calorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a cellular modulator of disaggregation activity or aggregation activity, or secondary antibodies that recognize the antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Finally simple calorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The following examples of specific embodiments for carrying out the present invention are offered for illustrative purposes only, and are not intended to limit the scope of the present invention in any way.

### EXEMPLARY EMBODIMENTS

### Example 1

### Reduced IIS Activity Lowers Aβ₁₋₄₂ Toxicity

One hypothesis to explain late onset aggregation-associated toxicity posits that the deposition of toxic aggregates is a stochastic process, governed by a nucleated polymerization, requiring many years to initiate disease. Alternatively, aging could enable constitutive aggregation to become toxic as a result of declining detoxification activities. To distinguish between these two possibilities, we asked what role the aging process plays in Aβ₁₋₄₂ aggregation-mediated toxicity in a C. elegans model featuring intracellular Aβ₁₋₄₂ expression. Link et al., Neurobiol Aging 22: 217, 2001. If Aβ₁₋₄₂ toxicity results from a non age-related nucleated polymerization, animals that express Aβ₁₋₄₂ and whose lifespan has been extended would be expected to succumb to Aβ₁₋₄₂ toxicity at the same rate as those with a natural lifespan. However, if the aging process plays a role in detoxifying an ongoing protein aggregation process, alteration of the aging program would postpone the initiation of aggregation-mediated toxicity. To examine these hypotheses, we used worms that express the human Aβ₁₋₄₂ minigene driven by the unc-54 promoter [strain CL2006; referred hereafter as Aβ₁₋₄₂ worms, (Link, Proc Natl Acad Sci USA 92: 9368, 1995)]. Accordingly, Aβ₁₋₄₂ is solely expressed within the body wall muscles resulting in their paralysis. Link, Proc Natl Acad Sci USA 92: 9368, 1995 The unaffected neighboring muscles serve as an intraorganismal control in the RNA interference (RNAi)-based experiments that alter aging.

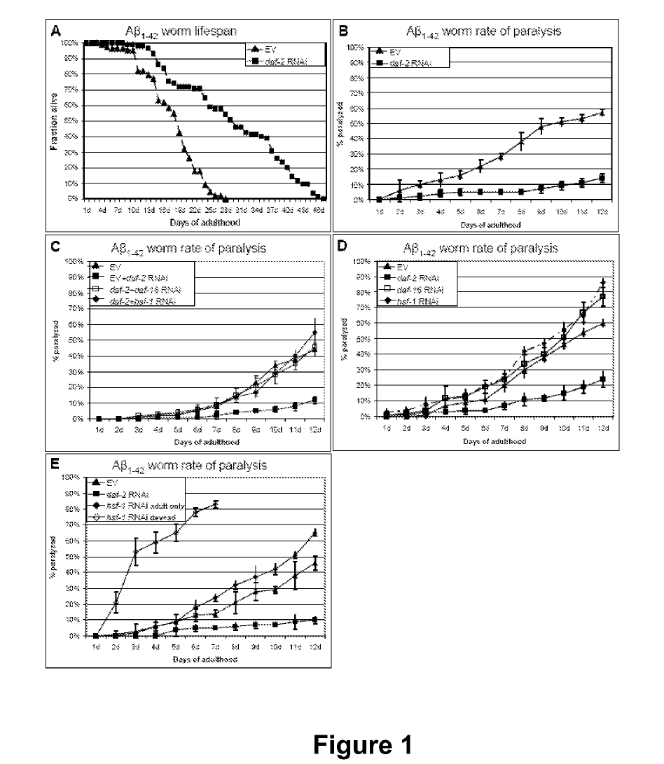

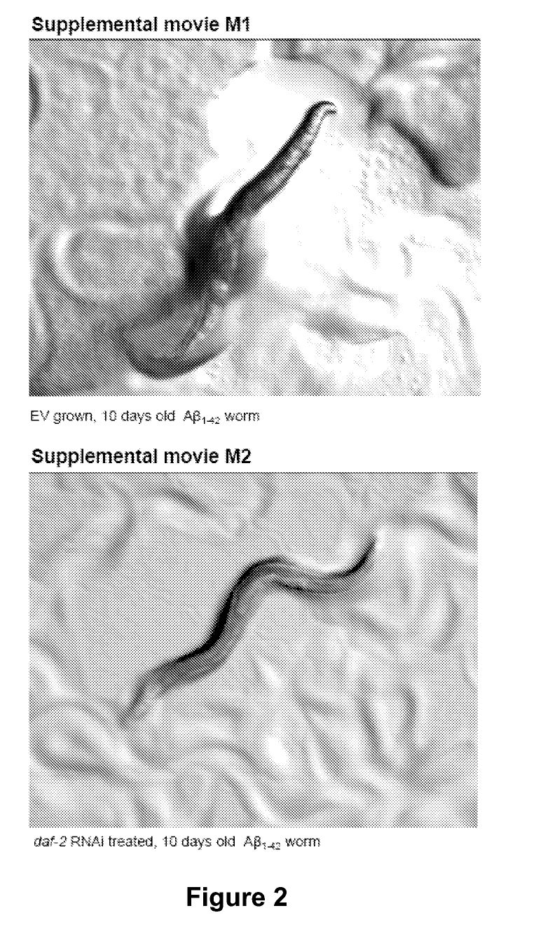

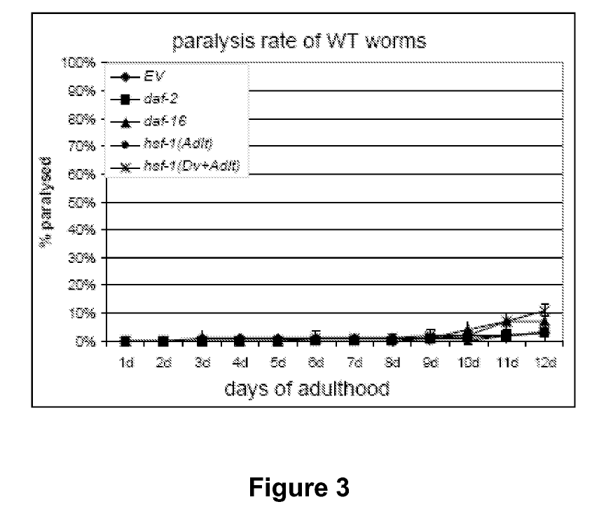

We first tested whether daf-2 RNAi extends the lifespan of Aβ₁₋₄₂ worms. Aβ₁₋₄₂ animals grown on potent daf-2 RNAi bacteria (Dillin et al., Science 298: 830, 2002) exhibited increased lifespan compared to control Aβ₁₋₄₂ worms grown on empty RNAi vector bacteria (EV) (FIG. 1A), similar to previous results with wild-type C. elegans. Kenyon et al., Nature 366: 461, 1993. daf-2 RNAi also remarkably decreased the fraction of paralyzed worms compared to control animals over a twelve-day time course (FIG. 1B; and FIG. 2). At day 10 of adulthood, 50% of the control worms were paralyzed compared to 10% of the daf-2 RNAi worms. C. elegans not expressing Aβ₁₋₄₂, showed no aging-related paralysis through 9 days and less than 7% after 12 days (FIG. 3). Collectively these data demonstrate that alteration of the aging program suppresses the pathological effect of Aβ₁₋₄₂ expression within the body wall muscles of C. elegans. Thus, Aβ₁₋₄₂ proteotoxicity does not appear to be a stochastic process, but it is highly dependent upon the aging process.

FIG. 1 shows ITS regulates proteotoxicity of Aβ₁₋₄₂ expressed in C. elegans. A. daf-2 RNAi extends lifespan of Aβ₁₋₄₂ worms. Aβ₁₋₄₂ worms were grown on bacteria expressing either the empty vector (EV) or daf-2 RNAi during development and adulthood. daf-2 RNAi worms lived significantly longer, p<0.0001, (squares, mean LS 28.6 days), than their EV grown counterparts (triangles, mean LS 17.8 days). B. daf-2 RNAi reduces Aβ₁₋₄₂ mediated toxicity. Aβ₁₋₄₂ worms were grown as in A. Numbers of paralyzed worms were scored daily for 12 days of adulthood. daf-2 RNAi (solid squares) reduced the number of paralyzed worms compared to the EV (solid triangles). C. Both daf-16 and hsf-1 RNAi abolish the protective effect of daf-2 RNAi towards Aβ₁₋₄₂ toxicity. Aβ₁₋₄₂ worms were grown during development and adulthood on EV bacteria (triangles), or on dilutions of equal amounts of bacteria expressing the following RNAi species: daf-2 and EV (solid squares), or daf-2 and daf-16 (open squares), or daf-2 and hsf-1 (solid diamonds). D. daf-16 or hsf-1 RNAi during adulthood results in an elevated rate of paralysis late in life. Aβ₁₋₄₂ worms were developed on EV and were transferred at day 1 of adulthood to bacteria expressing either: EV (triangles), daf-2 RNAi (solid squares), daf-16 RNAi (open squares) or hsf-1 RNAi (diamonds). daf-2 RNAi reduced the number of paralyzed animals, whereas both daf-16 and hsf-1 RNAi increased the number of paralyzed worms late in life compared to the EV. E. Reduced expression of hsf-1 during development (dev) and adulthood (ad) further accelerates the rate of paralysis. Aβ₁₋₄₂ worms were grown during development and adulthood on bacteria expressing either EV (solid triangles), daf-2 RNAi (solid squares) or hsf-1 RNAi (open diamonds), or were developed on EV and transferred to hsf-1 RNAi bacteria on day 1 of adulthood (solid diamonds).

FIG. 2 shows an EV grown Aβ₁₄₂ C. elegans worm and a daf-2 RNAi treated Aβ₁₋₄₂ C. elegans worm.

FIG. 3 shows minimal (less than 10%) paralysis of wild-type worms (not expressing Aβ₁₄₂) detected through day 12 of adulthood. Wild-type worms (strain N2) were grown on either EV, daf-2, daf-16 or hsf-1 RNAi bacteria, as indicated. Paralyzed worms were scored daily.

### Example 2

### DAF-16 and HSF-1 are Required for the Protective Effect of Reduced IIs

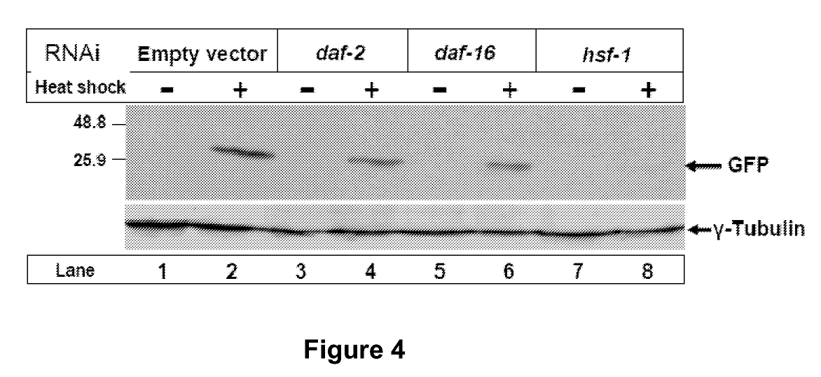

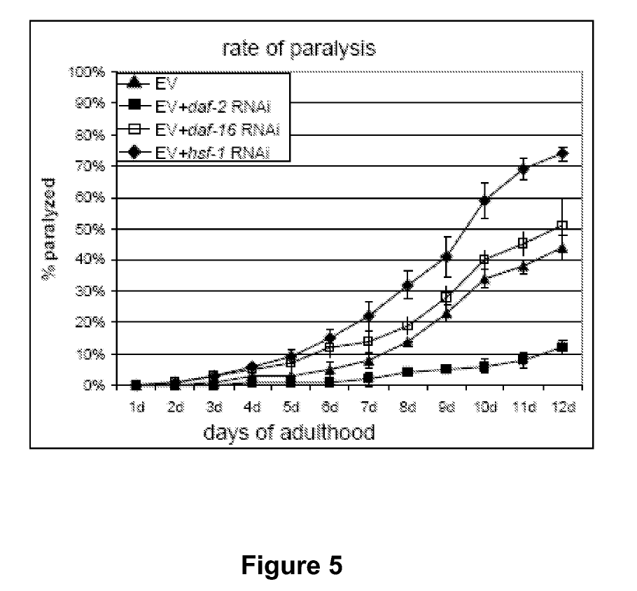

To understand the mechanism by which reduced IIS protected against Aβ₁₋₄₂ proteotoxicity, the question asked was whether daf-16 and/or hsf-1, genes that encode transcription factors necessary, but not sufficient, for the full extended lifespan of daf-2 mutant animals (10, 13), were also required for amelioration of proteotoxicity by daf-2 RNAi. Dilution of daf-2 RNAi bacteria with equal amounts of either effective hsf-1 (FIG. 4) or daf-16 (Dillin et al., Science 298: 830, 2002) RNAi bacteria abolished the daf-2 RNAi protective effect (FIG. 1C). A correspondingly equal dilution of daf-2, daf-16 or hsf-1 with the EV bacteria did not influence the paralysis phenotype of each RNAi treatment (FIG. 5). Therefore, analogous to the roles of daf-16 and hsf-1 in the regulation of aging by daf-2, both daf-16 and hsf-1 are necessary for the reduced IIS mediated amelioration of Aβ₁₋₄₂ proteotoxicity in the C. elegans body wall muscles.

Tests were performed to determine whether reduction of daf-16 or hsf-1 affected paralysis rates in animals with an intact daf-2. To avoid potential developmental disorders due to RNAi knockdown, we individually inactivated daf-2, daf-16 or hsf-1 only during adulthood, the period required for the IIS pathway to regulate the aging process. As before, daf-2 RNAi reduced the number of paralyzed worms (FIG. 1D). In contrast, either daf-16 or hsf-1 RNAi, increased the number of paralyzed animals after 8 days. Reduction of hsf-1 during development and adulthood increased the rate of paralysis relative to during adulthood only (FIG. 1E). In contrast, rates of paralysis of animals grown on daf-16 RNAi bacteria during development and adulthood versus adulthood only were very similar.

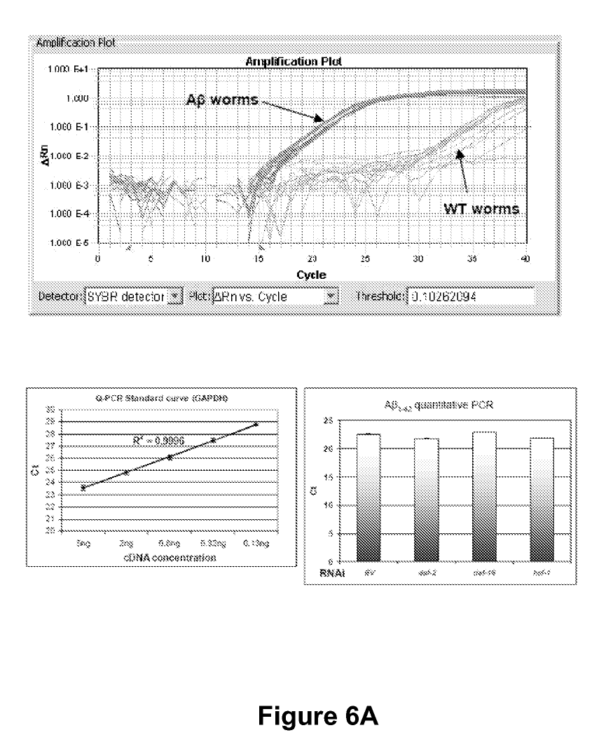

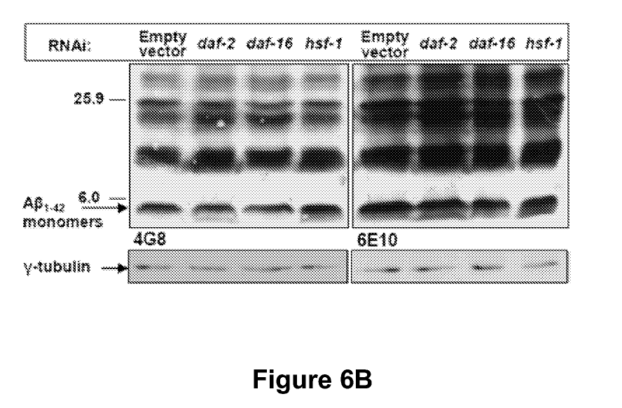

To evaluate whether RNAi utilization caused differential expression of Aβ₁₋₄₂ quantitative RT-PCR was performed. Quantitative RT-PCR experiments indicated that the levels of Aβ₁₋₄₂ mRNA transcripts within Aβ₁₋₄₂ worms grown on EV, daf-2, daf-16, or hsf-1 RNAi bacteria were nearly identical (FIG. 6A). In addition, western blot (WB) analysis revealed that soluble Aβ₁₋₄₂ levels were equivalent in all RNAi applications (FIG. 6B). Thus, the different levels of proteotoxicity cannot be explained by modulation of Aβ₁₋₄₂ expression.

Taken together, these results suggest that DAF-16 and HSF-1 target genes are jointly essential for the Aβ₁₋₄₂ detoxification mediated by inhibition of the IIS pathway. However, the HSF-1 transcriptome appears to play a more important role in suppressing Aβ₁₋₄₂ mediated toxicity, especially during development (FIG. 1E).

FIG. 4 shows hsf-1 RNAi bacteria effectively prevents induction of HSF-1 target gene expression. To assess the efficiency of hsf-1 RNAi bacteria we used a worm strain in which the reporter Green Fluorescent Protein (GFP) is driven by the HSF-1 regulated hsp-16.2 promoter (strain CL2070 (Kimura et al., Science 277: 942, 1997)) Worms were grown on either EV, daf-2, daf-16 or hsf-1 RNAi bacteria, heat stressed (33° C., 3 h) and subjected to WB analysis using GFP mAb. Unlike in worms grown on either EV, daf-2 or daf-16 RNAi bacteria, no GFP signal was detected in hsf-1 RNAi treated worms.

FIG. 5 shows dilutions daf-16 and of hsf-1 RNAi do not influence their toxic effect. Bacteria expressing daf-16 or hsf-1 were diluted with equal amounts of bacteria harboring the EV. The dilutions did not influence the rates of paralysis of Aβ₁₋₄₂ worms.

FIG. 6 shows A. Quantitative RT-PCR indicates that in all RNAi treatments, mRNA levels of Aβ₁₋₄₂ are nearly identical. Aβ₁₋₄₂ worms were grown on EV, daf-2, daf-16 or hsf-1 RNAi bacteria. Quantitative RT-PCR indicates that Aβ₁₋₄₂ is equally expressed in worms of all RNAi treatments. B. daf-2, daf-16 and hsf-1 do not directly affect the level of soluble Aβ₁₋₄₂. Aβ₁₋₄₂ worms were grown to adulthood on EV bacteria or daf-2, daf-16 or hsf-1 RNAi bacteria. WB analysis of Aβ using the mAb 4G8 or 6E10, detected no difference in total amount of soluble Aβ₁₋₄₂.

### Example 3

### High Molecular Weight Aβ₁₋₄₂ Aggregates do not Correlate with Toxicity

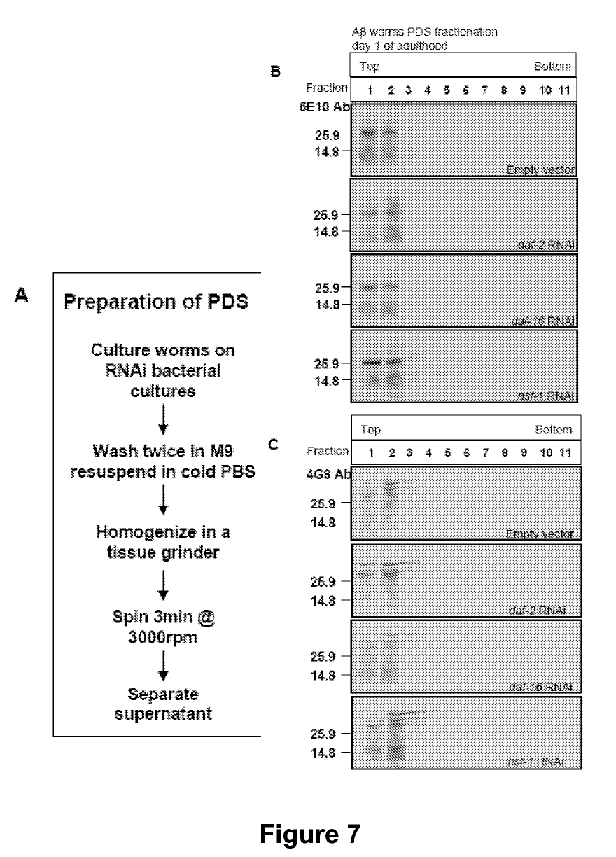

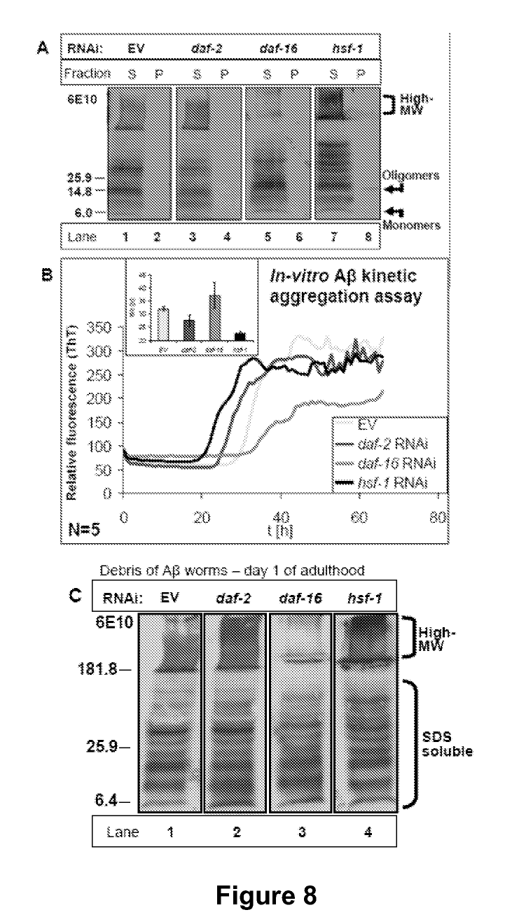

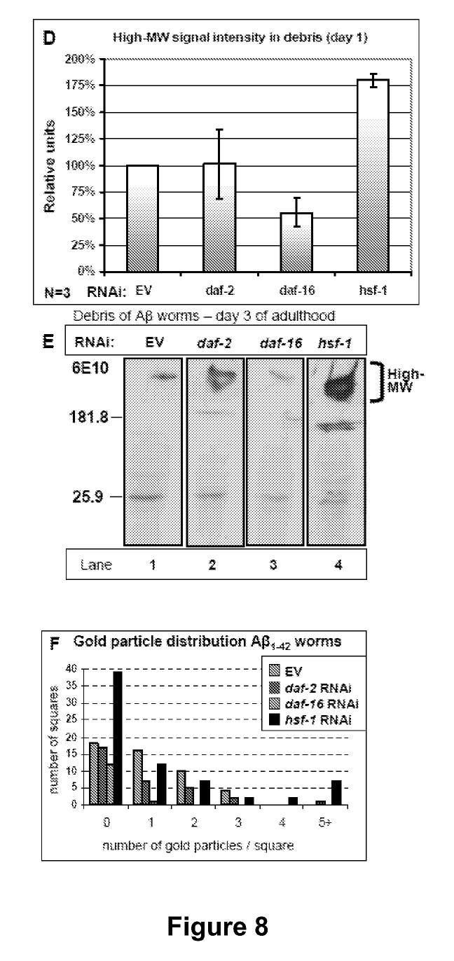

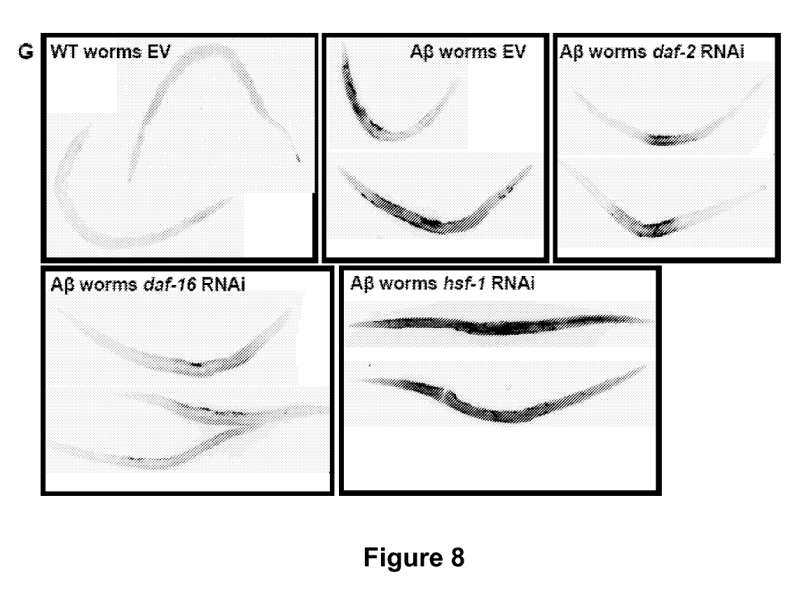

How could DAF-16 and HSF-1 protect worms from proteotoxicity? One possibility is that these transcriptomes prevent the formation of high-molecular weight (high-MW) aggregates that are linked to toxicity. Alternatively, these transcriptomes could detoxify smaller oligomers that mediate toxicity. Caughey and Lansbury, Annu Rev Neurosci 26: 267, 2003; Ross and Poirier, Nat Rev Mol Cell Biol 6: 891, 2005. To evaluate these possibilities, we asked whether daf-2 RNAi mediated protection from Aβ₁₋₄₂ proteotoxicity and suppression of this protective effect by knockdown of either daf-16 or hsf-1 expression, correlated with the amount of high-MW Aβ₁₋₄₂ aggregates. Aβ₁₋₄₂ worms were grown to day one of adulthood on EV or on daf-2, daf-16, or hsf-1 RNAi bacteria. The worms were homogenized, centrifuged for 3 min at 3000 rpm to afford pellet and post-debris supernatant fractions [PDS; (FIG. 7A)] that were evaluated separately (FIG. 7B, 7C). The PDS was subjected to ultracentrifugation revealing the presence of high-MW Aβ₁₋₄₂ aggregates (FIG. 8A). If high-MW Aβ₁₋₄₂ aggregates were the toxic species, then daf-2 RNAi animals would be expected to have less high-MW aggregates and daf-16 and hsf-1 RNAi animals would be expected to accumulate more. PDS from hsf-1 RNAi worms have the most high-MW aggregates, followed by daf-2 and EV RNAi, whereas in daf-16 RNAi worms the aggregates are hardly detectable. This observation is not consistent with high-MW Aβ₁₋₄₂ aggregates being the toxic species.

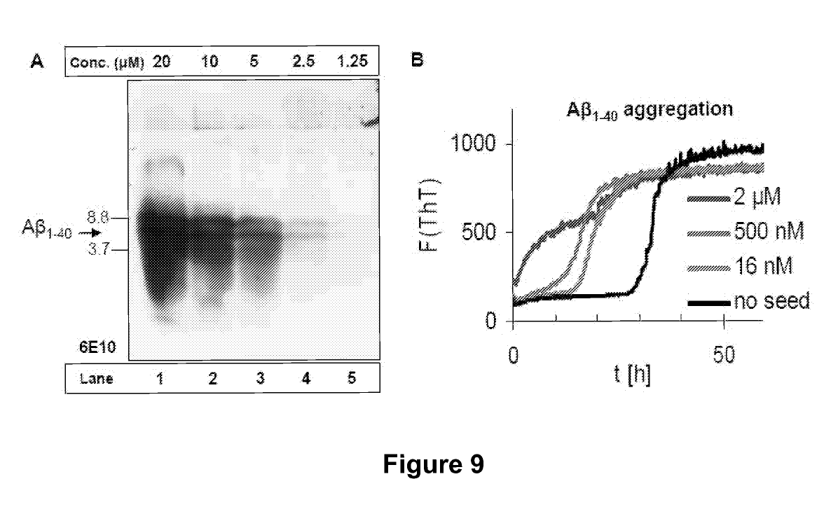

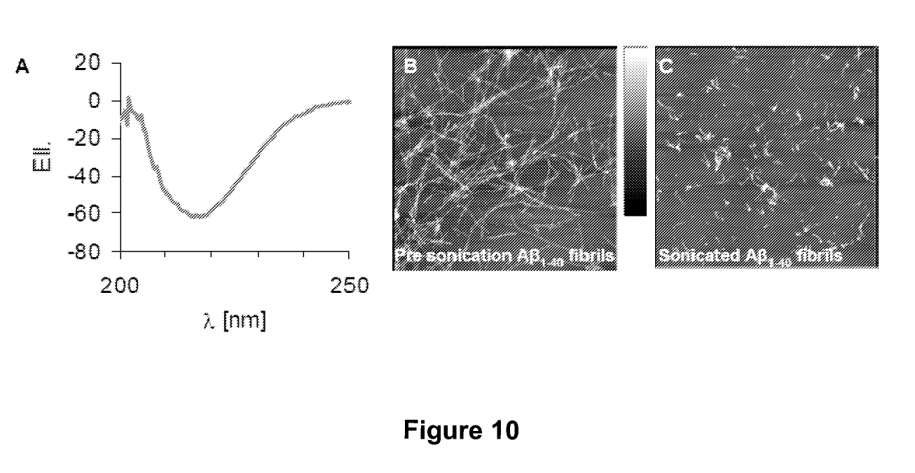

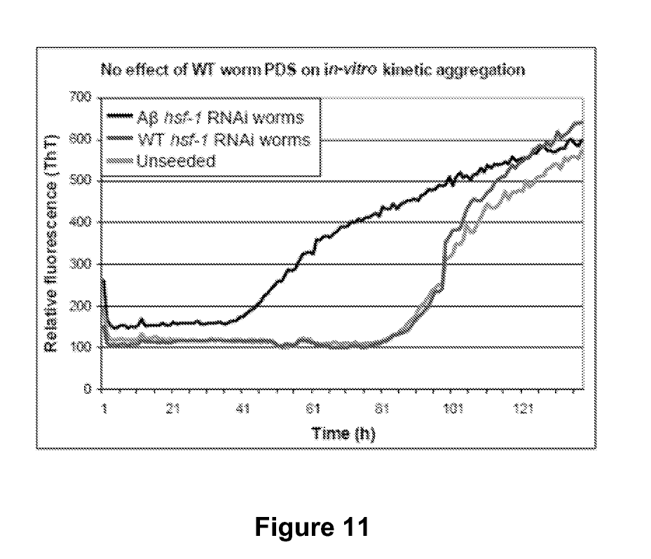

Aβ₁₋₄₂ aggregates in the PDS fraction were quantified with an in-vitro kinetic aggregation assay, which is at least three orders of magnitude more sensitive than WB analysis (FIG. 9). This assay enables the detection of small amounts of aggregates that can seed an Aβ₁₋₄₀ nucleated polymerization reaction in-vitro. Aβ₁₋₄₂ worm PDS was sonicated, to generate small fibrils (FIG. 10). Addition of sonicated PDS to an in-vitro Aβ₁₋₄₀ fibril formation assay would be expected to shorten the lag phase associated with the initiation of Aβ₁₋₄₀ aggregation, in a fashion that is dependent on the concentration of fibrillar seeds. Hasegawa et al., Biochemistry 38: 15514, 1999. PDS from Aβ₁₋₄₂ worms grown to day one of adulthood on EV, daf-2, daf-16 or hsf-1 RNAi bacteria were evaluated in-vitro by assessing the time for half maximal aggregation of Aβ₁₋₄₀ (t₅₀, FIG. 8B). Reactions seeded with PDS of hsf-1 RNAi worms had significantly (p<0.0002) faster Aβ₁₋₄₀ aggregation than the control worms (EV). Similarly, homogenates from daf-2 RNAi worms accelerated aggregation compared to the control worms (p<0.02), but less than PDS from hsf-1 RNAi worms. Finally, daf-16 RNAi worms harbored the least seeding competent aggregates. An in-vitro kinetic aggregation assay using hsf-1 RNAi worms not expressing Aβ₁₋₄₂ confirms that the lag phase shortening is completely dependent upon the presence of Aβ₁₋₄₂ (FIG. 11).

Within the worm debris less high-MW aggregates were found in the EV and daf-2 RNAi treated Aβ₁₋₄₂ worms compared to the increased amount found when hsf-1 was reduced. In contrast to hsf-1 RNAi treatment, the least amount of high-MW Aβ₁₋₄₂ aggregates were found in the debris of daf-16 RNAi worms (FIGS. 8C, 8D). These findings were extended beyond day one by growing the Aβ₁₋₄₂ worms to day three of adulthood and analyzing the worm debris by gel electrophoresis. More high-MW aggregates were present in debris from hsf-1 RNAi worms (FIG. 8E) compared to those grown on the EV, daf-2 or daf-16 RNAi bacteria. In contrast, the least high-MW Aβ₁₋₄₂ aggregates were detected in daf-16 RNAi worms. Formic acid extraction of all of the Aβ₁₋₄₂ worm debris prior to analysis also indicated that hsf-1 RNAi worms contained more high-MW Aβ₁₋₄₂ than daf-16 RNAi worms.

Importantly, the relative amounts of Aβ₁₋₄₂ aggregates observed in the PDS and in the worm debris analyzed by WB rank order identically, with hsf-1 RNAi worms having the most, followed by daf-2 RNAi, followed by the Aβ₁₋₄₂ worms grown on EV bacteria, followed by daf-16 RNAi worms.

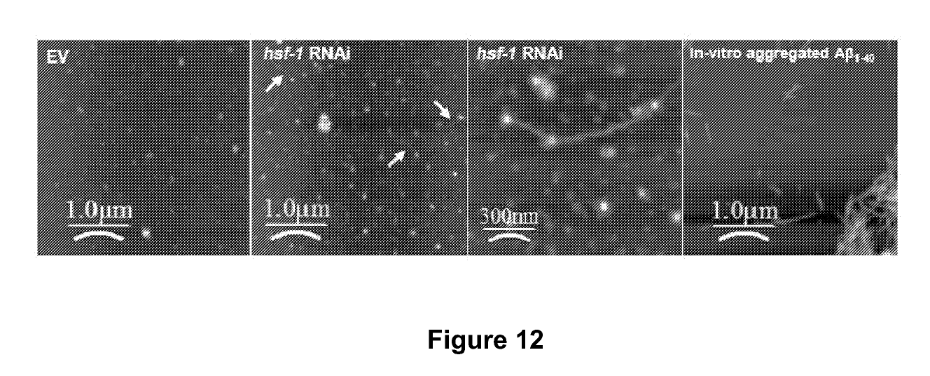

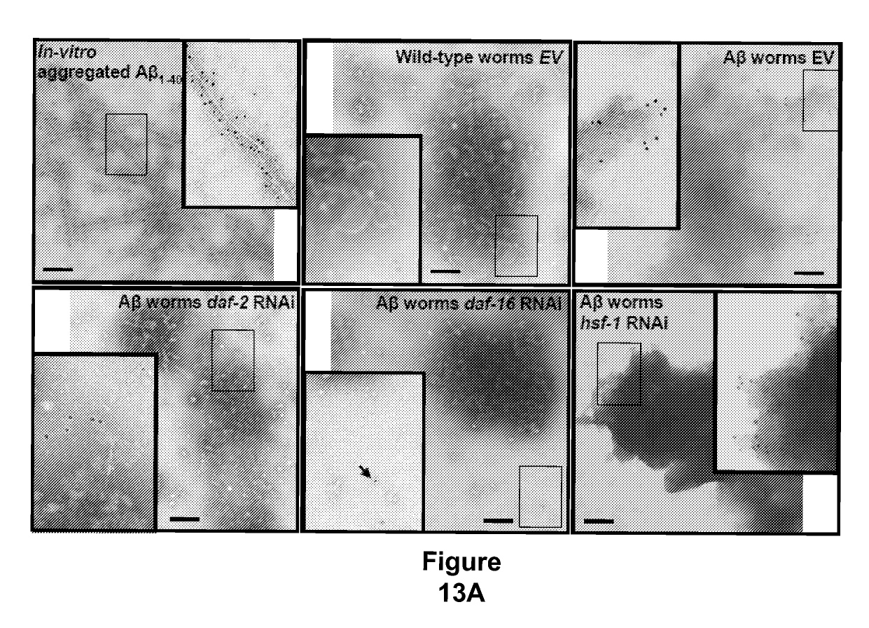

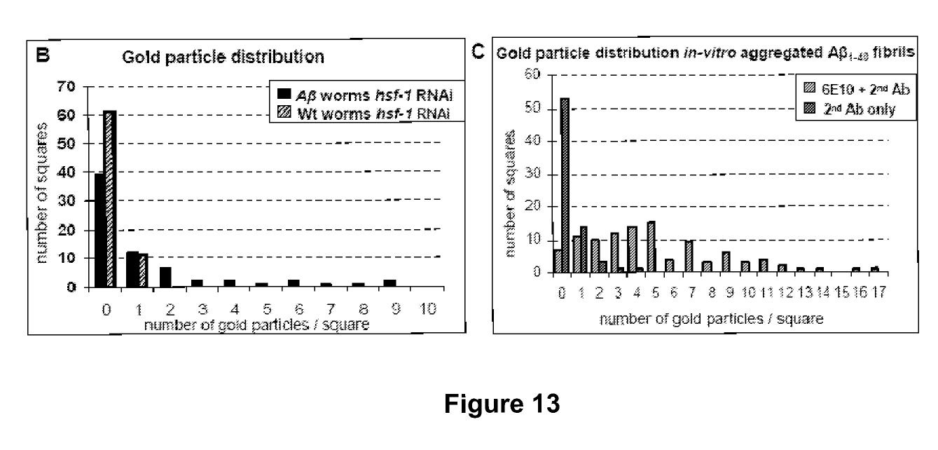

Using multiple ultracentrifugation steps with a final analysis by Atomic Force Microscopy (AFM), we directly visualized high-density material purified from Aβ₁₋₄₂ worm PDS. Fibrillar structures were detected only in the hsf-1 RNAi worms, but not in PDS of EV controls (FIG. 12), daf-2 or daf-16 RNAi worms. To verify that the fibrillar structures observed by AFM contain Aβ₁₋₄₂, we employed Immuno Electron Microscopy (FIG. 13A). Quantification and distribution analysis of the gold particles labeling Aβindicated that hsf-1 RNAi results in the most intense and specific signal. The PDS of Aβ₁₋₄₂ worms fed EV or daf-2 RNAi bacteria had a weaker signal, whereas the daf-16 RNAi worms had the least intense signal (FIG. 8F). Wild-type worms not expressing Aβ₁₋₄₂ that were fed hsf-1 RNAi bacteria (negative control, FIG. 13B) and in-vitro aggregated Aβ₁₋₄₀ (positive control, FIG. 13C) confirmed the immuno gold particle signal specificity. Finally, Aβ₁₋₄₂ worms were grown to day 2 of adulthood on the various RNAi species and Aβwas visualized within the intact worm using immuno-fluorescence (IF) microscopy. daf-2 RNAi and EV worms had similar intermediate intensities, hsf-1 RNAi treated Aβ₁₋₄₂ worms exhibited the most intense signal and daf-16 RNAi treatment resulted in the weakest signal (FIG. 8G).

Collectively, results from five independent methods studying worm debris, PDS, and intact worms all indicate hsf-1 RNAi worms contain the largest amount of fibrils and high-1-MW Aβ₁₋₄₂ aggregates, daf-2 RNAi worms contain slightly more than their EV treated counterparts, whereas daf-16 RNAi worms contain the fewest fibrils and high-MW Aβ₁₋₄₂ aggregates. This points to a lack of correlation between high-MW Aβ₁₋₄₂ aggregates and Aβ₁₋₄₂ mediated toxicity as (i) daf-2 RNAi reduces toxicity but slightly enhances the amount of high-MW aggregates, (ii) daf-16 RNAi increases toxicity but reduces the amount of high-MW aggregates and (iii) hsf-1 RNAi increases both toxicity and amount of high-MW Aβ₁₋₄₂ aggregates. The detection of aggregated Aβ₁₋₄₂ in early adulthood (days 1-2 of adulthood), prior to onset of paralysis (days 5-12) suggests that protein aggregation occurs early and throughout the life of the worm and is counteracted by aging-related processes to reduce toxicity.

FIG. 7 shows A. Schematic description of PDS preparation. B. Aβ₁₋₄₂ worms were fed bacteria expressing EV or dsRNA of daf-2, daf-16 or hsf-1 to day 1 of adulthood. The worms were homogenized and equal amounts of post debris supernatants (PDS, Fig. S5A) were fractionated through linear 10-60% sucrose gradients. Eleven gradient fractions were analyzed using the AβmAb 6E10. No highly aggregated Aβ₁₋₄₂ could be detected in PDS (bottom fractions). C. Blots of experiment S5B reported with the anti AβmAb 4G8.

FIG. 8 shows lack of correlation between Aβ₁₄₂ high-MW aggregates and toxicity. A. Aβ₁₋₄₂ worms were fed bacteria expressing EV or dsRNA towards of daf-2, daf-16 or hsf-1 to day 1 of adulthood. The worms were homogenized and equal amounts of post debris supernatants (PDS, Fig. S5A). Worms were homogenized and equal amounts of PDS were incubated for 30 min on ice with 1% Sarkosyl and spun for 1 h in an ultracentrifuge (427,000 g). Supernatants and pellets were separated and loaded onto denaturing 12% PAA gels and analyzed by WB using 6E10 mAb. No Aβ signal was detected in pellets of EV, daf-2 and daf-16 RNAi worm PDS (lanes 2, 4, and 6 respectively). A weak 16 kDa Aβband was detected in the pellet of PDS from hsf-1 RNAi worms (lane 8, oligomeric). B. RNAi of Aβ₁₋₄₂ worms as in A. PDS were prepared at day 1 of adulthood and were used to seed in-vitro kinetic Aβ₁₋₄₀ aggregation reactions that were monitored using Thioflavin-T (ThT) fluorescence. PDS of hsf-1 RNAi treated animals (black) exhibit the most significant acceleration of the reaction, indicating the most seeding competent aggregates. daf-2 RNAi (blue) accelerated the reaction compared to the EV (green) while daf-16 RNAi (red) had the least seeding competent aggregates. Inset: Statistical analysis of results obtained in B. C. Aβ₁₋₄₂ worms were grown and treated as in A. Debris were analyzed using 12% SDS PAA gel and WB. The largest amount of Aβ₁₋₄₂ high-MW aggregates was detected in animals with reduced hsf-1 (lane 4), daf-16 RNAi resulted in the least (lane 3) while daf-2 RNAi (lane 2) had slightly more Aβ₁₋₄₂ high-MW aggregates. D. Statistical analysis of the WB high-MW aggregate intensities in C. E. RNAi of Aβ₁₋₄₂ worms as in A. Aβcontents of the worm debris were analyzed at day 3 of adulthood using WB of a denaturing 4% PAA gel. The largest amount of high-MW Aβ₁₋₄₂ was detected in hsf-1 RNAi worms (lane 4), followed by daf-2 RNAi animals (lane 2), the least amount of Aβ₁₋₄₂ high-MW aggregates was found in daf-16 RNAi animals (lane 3). F. Quantification and distribution histogram analysis of gold particle labeling of the Aβ₁₋₄₂ worm preparations from immuno-EM analysis. EM-images (Fig. S10) were overlaid with a grid of 100×100 nm squares (see methods). Shown are distribution analyses of the number of immuno-gold particles found within squares that contained aggregate surfaces. The Aβ₁₋₄₂ immuno-gold staining signal of hsf-1 RNAi worms is the most intense while signal of Aβ₁₋₄₂ in daf-16 RNAi treated worms is the weakest. G. Aβ₁₋₄₂ worms were grown on RNAi bacteria as in A, to day 2 of adulthood and were subjected to immunofluorescence microscopy using the anti Aβ3 mAb 4G8. The signal intensity of daf-2 RNAi worms was similar to EV worms. daf-16 RNAi animals had the weakest signal and hsf-1 RNAi animals showed considerably stronger signal intensity. Wild-type animals not expressing Aβ₁₋₄₂ did not exhibit immunofluorescence signal, demonstrating the specificity of the Aβ₁₋₄₂ monoclonal antibody.

FIG. 9 shows the in-vitro kinetic aggregation assay is at least three orders more sensitive than WB. A. Dilution series of Aβ₁₋₄₀ as detected by WB (6E10, 1:5000, 20 μl/well). B. Aβ₁₋₄₀ (20 μM) aggregation assays were performed as in FIG. 8B in the presence of pre-aggregated and sonicated Aβ₁₋₄₀ as shown in supplemental FIG. 10.

FIG. 10 shows sonication disrupts large Aβ₁₋₄₀ fibrils into smaller fibrils. In-vitro synthesized Aβ₁₋₄₀ (1009M) was aggregated for 96 h at 37° C. A. Circular dichroism spectrum indicating that the Aβpeptide has largely adopted a β-sheet conformation, B. AFM image before and C. after sonication for 30 min. Height scale bar 20 nm.

FIG. 11 shows the lag phase shortening associated with seeding of in-vitro kinetic aggregation assay completely depends upon the presence of Aβ₁₋₄₂. PDS of hsf-1 RNAi grown wild-type or Aβworms were used to seed in-vitro kinetic aggregation assay. Aggregation kinetics of Aβ₁₋₄₀ in the presence of PDS from wild-type worms (blue) not expressing Aβ₁₋₄₂ and unseeded reaction (red) are indistinguishable, while seeding with PDS of Aβ₁₋₄₂ worms (black) dramatically shortened the time needed for aggregation to start.

FIG. 12 shows AFM images of ultracentrifugation pellets from PDS of EV grown, hsf-1 RNAi treated worms and in-vitro aggregated Aβ₁₋₄₀. Fibrillar aggregates were found exclusively in images (n=6) of hsf-1 RNAi treated animals (arrows) but not in images (n=9 of each) of daf-2, daf-16 or EV RNAi treated animal preparations (not shown). Likewise, AFM images of preparations from wild-type animals (strain N2) which do not express Aβ₁₋₄₂ do not show any fibrillar aggregates (n=6) regardless whether they were treated with hsf-1 or EV RNAi (not shown). Fibrils (n=12) were analyzed for morphology and had an average length of 500±200 nm an average height of 5±1 nm and a periodicity of 60±10 nm.

FIG. 13 shows immuno electron microscopy of Aβ₁₋₄₂ worm samples prepared as in FIG. 8A, indicate that preparations from hsf-1 RNAi treated worms contain the most immunoreactive Aβ₁₋₄₂ while daf-16 treated worm preparations contain the least. A. The samples were incubated with 6E10 Aβ3 mAb, and gold particle (10 nm) labeled secondary antibody, were negatively stained, and were viewed using electron microscopy (bar=200 nm). B. Comparison of Aβ₁₋₄₂ signal of wild-type (not expressing Aβ₁₋₄₂, black bars) and of Aβ₁₋₄₂ worm (dashed bars) preparations confirms the signal specificity. C. Distribution analysis of gold labeling in immuno-EM images of in-vitro aggregated Aβ₁₋₄₀ fibrils confirmed the specificity of 6E10 and secondary gold labeled antibody (red bars). Blue bars represent signal intensity and distribution when only secondary antibody was used.

### Example 4

### HSF-1, but not DAF-16, Controls Disaggregation of Aβ₁₋₄₂ Aggregates

Our findings suggest that two opposing mechanisms, regulated by the IIS pathway, protect worms from Aβ₁₋₄₂ mediated toxicity: the HSF-1 transcriptome regulates disaggregation whereas the DAF-16 transcriptome mediates the formation of less toxic high-MW aggregates. The latter is analogous to the aggregation-mediated neuroprotection included in Huntington's disease. Arrasate et al., Nature 431: 805, 2004; Saudou et al., Cell 95: 55, 1998. Both activities appear to be required to protect the Aβ₁₋₄₂ worms from early onset paralysis associated with proteotoxicity. Inhibiting the HSF-1 transcriptome during worm development is more deleterious than DAF-16 towards Aβ₁₋₄₂ toxicity, suggesting the former is the more important pathway (FIG. 1E). Thus, it is plausible that the HSF-1 controlled disaggregation and degradation pathway is the preferred pathway, whereas the DAF-16 controlled active aggregation is the backup pathway used mainly under severe stress conditions. The HSF-1 controlled disaggregation pathway in the Aβ₁₋₄₂ worms may be constantly overloaded. Thus, the DAF-16 regulated active aggregation machinery is continually assisting. Accordingly, daf-2 RNAi worms have slightly more high-MW aggregates than EV animals because both the HSF-1 regulated and the DAF-16 regulated pathways are fully active.

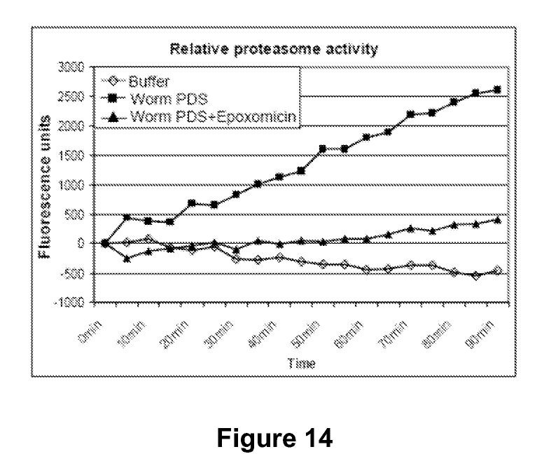

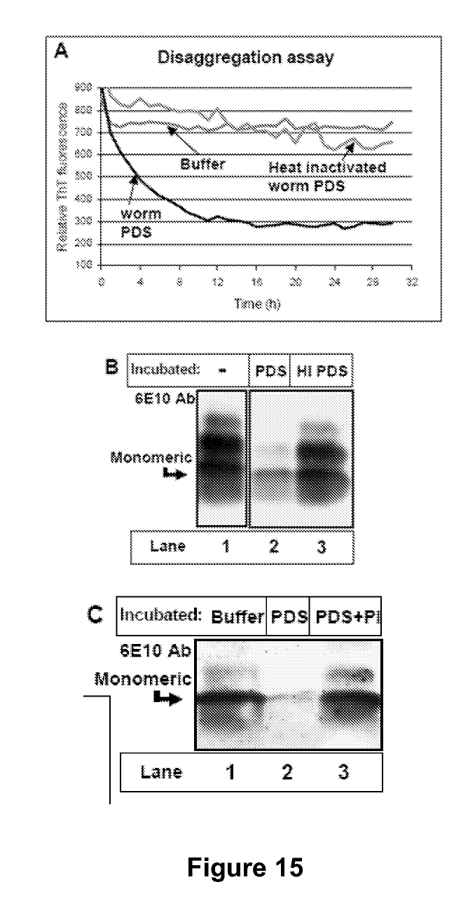

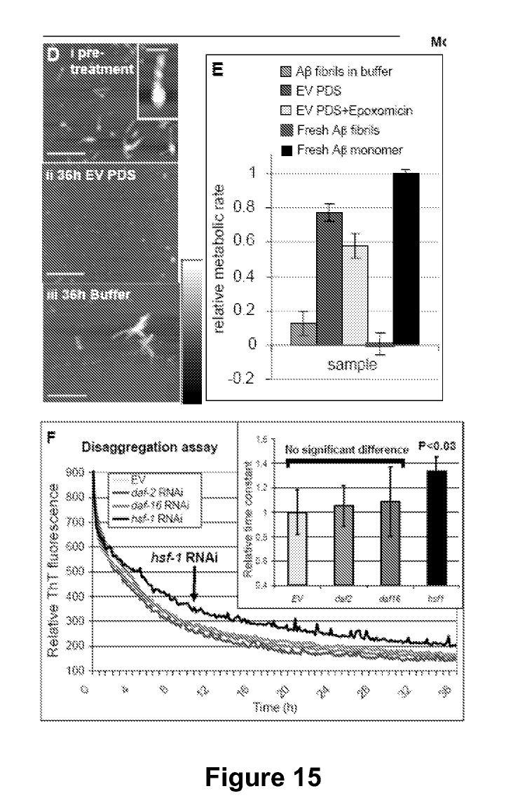

To test the hypothesis that Aβ₁₋₄₂ disaggregation in the worm is regulated by HSF-1, but not DAF-16, we developed an in-vitro assay to measure disaggregation of Aβ₁₋₄₀ fibrils by worm PDS. Time dependent disaggregation of Aβ₁₋₄₀ fibrils in-vitro, in the presence and absence of worm PDS was quantified. To exclude the possibility of monitoring proteasomal and/or proteolytic degradation, rather than disaggregation, experiments were performed in the presence of (i) the proteasome inhibitor Epoxomicin (10 μM) (FIG. 14) or (ii) protease inhibitor cocktail. In buffer alone (FIG. 15A) and with added BSA (0.5 mg/ml), the fibrils were stable for at least 36 h. In contrast, Aβ₁₋₄₀ fibrils underwent disaggregation when treated with PDS from Aβ₁₋₄₂ worms. Heat-inactivation of PDS (80° C., 20 min) destroyed the disaggregation activity (FIG. 15A). The amount of Aβ₁₋₄₀ detected by WB after 17 and 96 h of incubation with PDS, in the absence of protease inhibitors was reduced, indicating that Aβ₁₋₄₀ was proteolyzed subsequent to disaggregation. In contrast, heat-inactivated PDS had no detectable effect on the amount of Aβ₁, 40 (FIG. 15B). In the presence of a cocktail of protease inhibitors, Aβ₁₋₄₀ fibrils were disassembled but not degraded (FIG. 15C). Proteasome inhibitor alone did not prevent proteolysis, although more detailed experiments are needed before excluding proteasome involvement. Aβ₁₋₄₀ fibrils prepared in-vitro were visible by AFM before treatment with worm PDS and after a 36 h incubation with buffer, but not after incubation with PDS of EV treated Aβ₁₋₄₂ worms (FIG. 15D). Collectively, the results confirm that the assay detects disaggregation activity of worm homogenates, and demonstrate that worm PDS also proteolyses Aβ₁₋₄₀, yet, we can dissociate these activities with the appropriate use of protease inhibitors. In the presence of protease inhibitors, Aβ₁₋₄₀ fibrils will spontaneously reform if a longer time window (20-40 h) is observed.

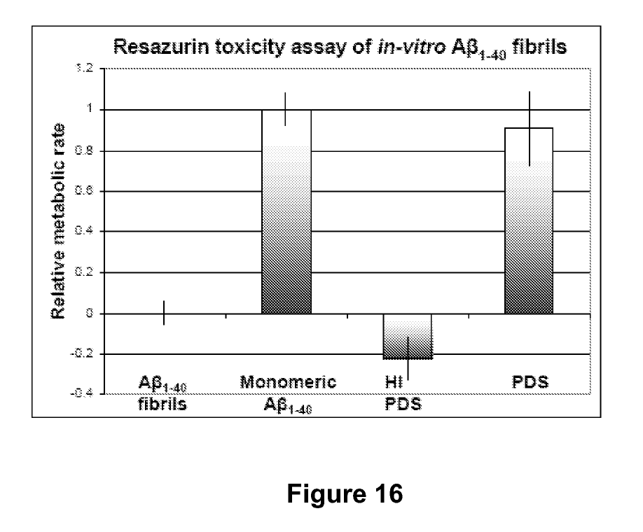

Does the disaggregation activity found within worm PDS reduce the toxicity of Aβ₁₋₄₀ fibrils? Viability of rat adrenal pheochromocytoma (PC12) cells were discerned by the MTT assay (Bucciantini et al., Nature 416: 507, 2002), and normalized to cell survival when incubated in the absence of Aβ₁₋₄₀. Worm PDS dramatically reduced cytotoxicity of Aβ₁₋₄₀ aggregates prepared in-vitro (FIG. 15E). Analogous results were obtained when Resazurin (O'Brien et al., Eur J Biochem 267: 5421, 2000) was used to measure Aβ₁₋₄₀ fibril toxicity on PC12 cells (FIG. 16). Thus, disaggregation directly correlates with detoxification of Aβ₁₋₄₀ fibrils in these widely used cell-based assays.

While HSF-1 regulated disaggregation activity could increase toxicity by releasing small, toxic aggregates, from larger less toxic aggregates, our data clearly point to the protective activity of HSF-1 suggesting a tight mechanistic link between disaggregation and degradation, possibly mediated by proteases such as the Insulin Degrading Enzyme (Leissring et al., Neuron 40: 1087, 2003) or Neprilysin. Iwata et al., Science 292: 1550, 2001.

We measured the disaggregation activity of PDS from Aβ₁₄₂ worms grown on either EV, daf-2, daf-16 or hsf-1 RNAi bacteria (FIG. 15F). Disaggregation curves were fit to an exponential decay function to quantify the results and to assess their statistical significance. No significant difference was observed among PDS of EV, daf-2 and daf-16 RNAi worms. However, PDS of hsf-1 RNAi worms exhibited a decreased disaggregation rate (35%, N=3, p<0.03), indicating that HSF-1 regulates the disaggregation activity (FIG. 15F, inset). Nevertheless, the relatively small effect of hsf-1 RNAi on disaggregation is surprising given its robust physiological response on Aβ₁₋₄₂ toxicity. One possibility is that HSF-1 also regulates protective functions other than disaggregation. Alternatively, HSF-1 may be one component in a more complex mechanism that regulates disaggregation. It is also possible that the 35% decline in disaggregation results in exacerbated Aβ₁₋₄₂ proteotoxicity. In any case, reduced hsf-1 slowed disaggregation whereas reduced daf-16 did not, supporting the notion that HSF-1 regulates disaggregation.

FIG. 14 shows 10 μM Epoxomicin effectively inhibits proteasome activity of worm PDS. The synthetic proteasome substrate Z-GGL-AMC was used according to the manufacturer's (Calbiochem) instructions to follow proteasome activity of wild-type worms (strain N2) PDS (0.5 μg/μl).

FIG. 15 shows hsf-1 is required for efficient disaggregation of Aβ₁₋₄₂ aggregates. A. Pre-aggregated, Thioflavin-T labeled Aβ₁₋₄₀ fibrils were incubated with either buffer (green), Aβ₁₋₄₂ worm PDS (black) or heat inactivated PDS (red) in the presence of Epoxomicin (10 μM). ThT fluorescence emission declined in the presence of worm PDS, indicating disaggregation activity. The Aβ₁₋₄₀ fibrils were stable in both buffer and heat inactivated PDS. B. Pre- and post-disaggregation samples were loaded onto 10% PAA gel. Api-40 was visualized by WB using 6E10, before the reaction (lane 1), after 96 h in the presence of worm PDS (lane 2) or in the presence of heat inactivated PDS (lane 3). Less Aβ₁₋₄₀ was observed after incubation with worm PDS, but not with heat inactivated PDS. No proteasome inhibitors were used in this experiment. C. Disaggregation reaction was performed in the absence or presence of a protease inhibitor cocktail (PI) (lane 2 and 3, respectively). WB analysis indicated that in the presence of PI, the total quantity of Aβ₁₋₄₀ did not change compared to the buffer incubated fibrils despite the disaggregation. D. In-vitro aggregated Aβ₁₋₄₀ fibrils were visualized using AFM, with no treatment (i), after 36 h incubation with EV-grown Aβ₁₋₄₂ worm PDS (ii), and after a 36 h incubation with buffer only (iii). No large fibrils were detected after incubation with worm PDS. All large bars represent 1 μm, inset bar 200 nm, height scale bar 20 nm. E. Worm disaggregation activity reduces the Aβ₁₋₄₀ fibril mediated cytotoxicity in cell based assays. Using the disaggregation assay and conditions as in B, and 72 h incubation, Aβ₁₋₄₀ disaggregation samples (500 nM) were added to PC12 cell culture medium for 3 days. Cell viability was assayed by MTT metabolic activity. Bucciantini et al., Nature 416: 507, 2002. Aβ₁₋₄₀ toxicity was reduced in samples incubated with worm PDS in the presence (green) or absence (blue) of Epoxomicin (10 μM). Samples incubated without worm PDS (red) showed similar toxicity to the starting material (purple). Monomeric Aβ₁₋₄₀ peptide did not exhibit toxicity under the assay conditions (black). Similar results were found using a Resazurin based assay (FIG. 16). F. hsf-1 is required for efficient disaggregation of pre-formed Aβ₁₋₄₀ fibrils. RNAi of Aβ₁₋₄₂ worms as in 2A. PDS of hsf-1 RNAi worms (black) exhibited 20-50% decline in disaggregation activity compared to PDS of EV worms (green). No significant change in disaggregation activities in PDS of daf-2 (blue) or daf-16 (red) RNAi worms. Inset: Statistical analysis of disaggregation results shown in F indicate that EV and hsf-1 RNAi worms are significantly (p<0.03) different (n=3).

FIG. 16 shows resazurin assay (Kenyon, Cell 120: 449, 2005) indicates that disaggregation activity possessed by worm homogenate reduces the toxicity of in-vitro aggregated Aβ₁₋₄₀ fibrils on PC12 cells (assay conditions as in FIG. 15E). This protective effect was abolished by heat inactivation of the homogenate.

### Example 5

### Small Aβ₁₋₄₂ Oligomers Correlate with Toxicity

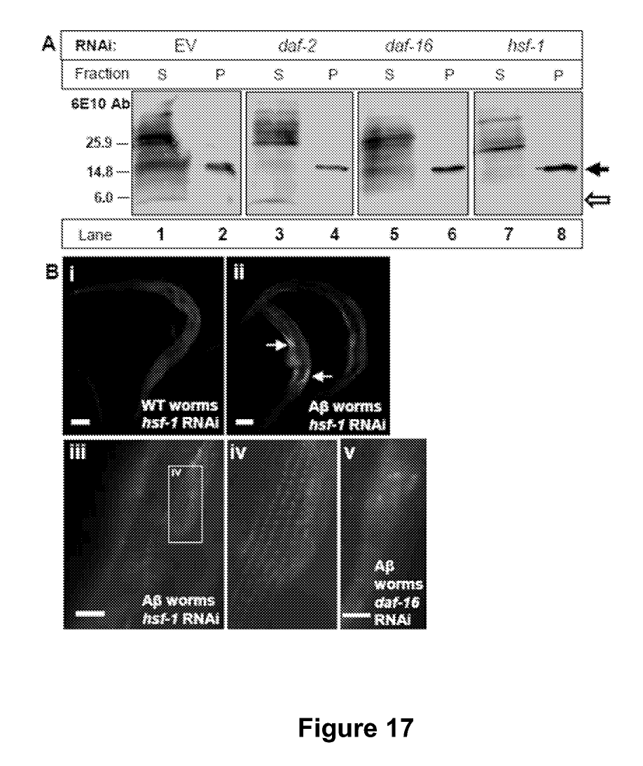

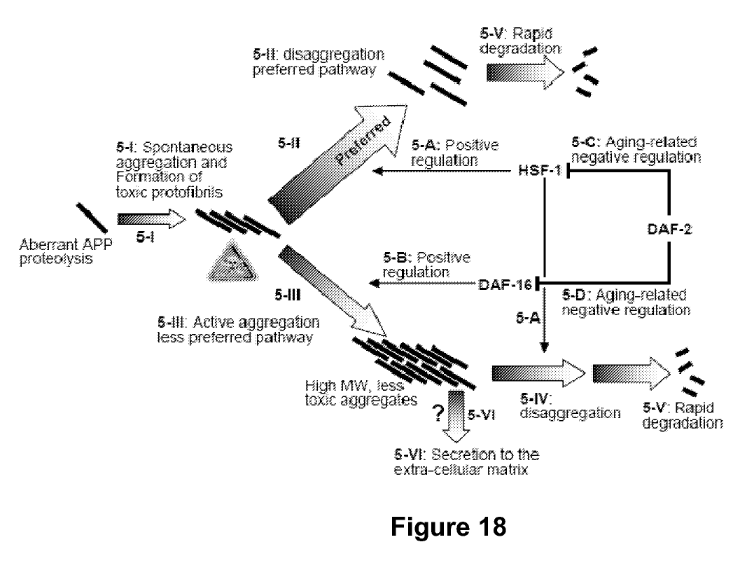

The lack of correlation between proteotoxicity and large-MW aggregates suggests that small oligomers may be the key toxic species in the worm Aβ₁₋₄₂ aggregation model. Numerous lines of evidence implicate Aβ₁₋₄₂ oligomers in proteotoxicity. Gong et al., Proc Natl Acad Sci USA 100: 10417, 2003; Hartley et al., J Neurosci 19: 8876, 1999; Walsh et al., Nature 416: 535, 2002. To determine whether small Aβ₁₋₄₂ aggregates correlate with toxicity, worms were grown to adulthood on EV, daf-2, daf-16 or hsf-1 RNAi bacteria and the PDS fractions were subjected to ultracentrifugation and the soluble supernatants and insoluble pellets analyzed by WB (FIG. 8A). No Aβwas detected in the insoluble pellets of EV, daf-2 and daf-16 RNAi treated worms. However, a weak Aβimmuno reactive band of approximately 16 kDa was detected in the insoluble pellet of hsf-1 RNAi worms (FIG. 8A). This band size is consistent with a SDS-stabilized Aβ₁₋₄₂ trimer, possibly derived from a larger quaternary structure. When PDS incubation with 1% Sarkosyl was shortened to 10 min, the 16 kDa protein was detected in all insoluble pellets (FIG. 17A). Notably, the amounts of the 16 kDa species correlated with Aβ₁₋₄₂ toxicity; insoluble pellets from daf-2 RNAi worms had the least intense signal, whereas daf-16, hsf-1 and EV RNAi worms had greater signal intensities. Aβ₁₋₄₂ monomers were detected in the supernatants of EV and daf-2 RNAi worms, but not in daf-16 and hsf-1 RNAi worms (FIG. 17A), suggesting that Aβ₁₋₄₂ monomers had formed trimers and/or larger assemblies thereof. In the absence of detergents, all of the Aβ₁₋₄₂ was retained in the insoluble pellet, suggesting that it may be associated with membranes. These observations are consistent with an Aβ₁₋₄₂ quaternary structure; apparently a trimer (16 kDa) or an oligomer thereof, mediates proteotoxicity, possibly in association with a membrane.

Using high resolution IF (FIG. 17B), Aβ₁₋₄₂ aggregates were detected along muscle fibers of Aβ₁₋₄₂ worms grown on either hsf-1 or on daf-16 RNAi bacteria. Only a small amount of Aβ₁₋₄₂ aggregates were detected along muscular fibers of Aβ₁₋₄₂ worms grown on EV and no such signal was detected in daf-2 RNAi worms. These results support the hypothesis that small Aβ₁₋₄₂ oligomers in spatial proximity to the muscle fibers correlate with toxicity in this worm model and are consistent with recent studies showing that Aβoligomers, potentially trimers, posses the highest toxicity towards hippocampal long term potentiation of neural cells (Townsend et al., J Physiol, 572: 477-492, 2006) and that Aβtrimer assemblies are involved in memory impairment of transgenic AD model mice. Lense et al., Nature 440: 325, 2006.

FIG. 17 shows intensity of an Aβ immuno-reactive 16 kDa band correlates with toxicity. A. Worm homogenates were prepared as in FIG. 8A, except the PDS was incubated for 10 mins on ice in 1% Sarkosyl to maintain more proteins in their membrane associated state. 16 kDa Aβbands were detected in all pellets (lanes 2, 4, 6 and 8, solid arrow). The most intense 16 kDa bands were seen in the pellet of daf-16 or hsf-1 RNAi worms (lanes 6 and 8 respectively) the least amount was found in the pellets of daf-2 RNAi worms (lane 4). Aβmonomers (˜5 kDa, open arrow) could be seen in supernatants of EV and daf-2 RNAi worms (lanes 1 and 3 respectively) but not in supernatants of daf-16 or hsf-1 RNAi worms (lanes 5 and 7 respectively). B. High resolution immuno-fluorescence microscopy using 4G8 Aβ3 mAb indicates that muscles are labeled in hsf-1 RNAi Aβ₁₄₂ worms (panel ii, arrows) but not in wild type worms (panel i). Small Aβ₁₋₄₂ aggregates were detected along the muscular fibers of worms grown on hsf-1 (panels iii and iv) and daf-16 RNAi (panel v). (In i and ii bar=75 μm, iii and v bar=15 μm).

### Example 6

### Model of DAF-16 and HSF-1 Mediated Protection Against Aβ₁₋₄₂ Aggregate Proteotoxicity

Our data suggests the following model for how daf-2 regulated pathways reduce aggregate mediated proteotoxicity (FIG. 18). First, (5-I) aggregation-prone peptides (such as Aβ₁₋₄₂) form small toxic aggregates constitutively. We suggest that cells have developed two mechanisms to detoxify these toxic intermediate aggregates. The preferred detoxification route is to efficiently disaggregate the toxic oligomer and degrade the amyloidogenic peptide (5-II), a pathway that is positively regulated by HSF-1 (5-A), perhaps via a subset of molecular chaperones. Muchowski and Wacker, Nat Rev Neurosci 6:11, 2005. When this pathway is overtaxed, another activity transforms toxic low-MW oligomers into high-MW aggregates of lower toxicity. This mechanism (5-III) is positively regulated by DAF-16 (5-B), through its target genes. The cell ultimately has to get rid of these large aggregates by either degrading them using the HSF-1 controlled disaggregation/degradation machinery (5-IV, 5-V) or, possibly, by their secretion (5-VI). The opposing disaggregation (HSF-1) and aggregation (DAF-16) detoxification pathways both are negatively regulated by IIS signaling (5-C, 5-D).

How does this model explain our results? (i) When hsf-1 is reduced, disaggregation in stages 5-II and 5-IV is impaired. This leads to a reduction of disaggregation of small toxic aggregates and leaves the cell no alternative but to actively form less toxic, high-MW aggregates, using the DAF-16 regulated machinery (5-III). Moreover, the absence of HSF-1 also appears to slow the clearance of high-MW aggregates (5-IV). Together, this leads to maximal accumulation of high-MW aggregates. (ii) When DAF-16 is reduced, cells lack the protective large aggregate formation machinery, resulting in higher toxicity. Yet, the cells can disaggregate and degrade small aggregates (5-II), resulting in less high-MW aggregates. The secondary importance of the DAF-16 regulated machinery explains why the toxicity of daf-16 RNAi is lower than that of hsf-1 RNAi. (iii) Knockdown of daf-2, a negative regulator of DAF-16 (5-D) and perhaps of HSF-1 (5-C) results in the upregulation of both protective mechanisms: (stages 5-II and 5-III). In this situation, both the clearance rate of small toxic aggregates and their detoxification by active aggregation are maximal, resulting in minimal toxicity. According to our model, the aging process actively reduces the cellular ability to detoxify small toxic aggregates by negative regulation of both detoxification mechanisms via the insulin like signaling pathway.

It is notable that the ITS pathway plays a role in modulating other forms of toxic protein aggregation, such as in the aggregation of the Huntington protein (Hsu et al., Science 300: 1142, 2003; Morley et al., Proc Natl Acad Sci USA 99: 10417, 2002; Parker et al., Nat Genet 37: 349, 2005) suggesting that the activities identified here may be quite general. Additionally, small perturbations of proper protein folding homeostasis have been demonstrated to have a profound impact on organismal integrity (Gidalevitz et al., Science 311: 1471, 2006), suggesting that the protective mechanisms regulated by the IIS pathway may link longevity to protein homeostasis.

FIG. 18 shows a model of age regulated HSF-1 and DAF-16 opposing anti-proteotoxicity activities. (5-I) Aggregation-prone peptides spontaneously form small toxic aggregates. (5-II) Specialized cellular machinery identifies toxic aggregates, rapidly disaggregates and prepares them for degradation. The products of this machinery are rapidly degraded (5-V). This preferred mechanism is positively regulated by HSF-1 (5-A) and negatively regulated by DAF-2 (5-C). (5-III) When HSF-1 regulated, disaggregation machinery is overloaded, a secondary machinery that mediates aggregation is activated forming less toxic high-MW aggregates. This machinery is positively regulated by DAF-16 (5-B) and negatively by DAF-2 (5-D). The high-MW aggregates, which accumulate as a result of the DAF-16 regulated mechanism, undergo either slow disaggregation and degradation by the HSF-1 regulated mechanism (5-IV and 5V) or possibly secreted to the extra-cellular matrix (5-VI).

### Example 10

### Experimental Methods

Protein Determination. Protein concentration was determined using a BCA kit (Pierce #23223). Epoxomicin (#324800) and the proteasome substrate Z-GGL-AMC (#539144) were purchased from Calbiochem (San Diego, Calif.). Complete protease inhibitor cocktail (#1836170) was from Roche (Basel, Switzerland). Aβ1-40 was purchased from Synpep (Dublin, Calif.). All other materials were from Sigma.

Antibodies. Aβmonoclonal antibodies clone 4G8 (9220) and clone 6E10 (9320) were from Signet (Dedham, Mass.). Anti γ-tubulin antibody clone GTU-88 (T-6557) was from Sigma. Secondary antibodies conjugated to HRP and to gold were purchased from Jackson Immuno-Research (West Grove, Pa., USA) or from Pierce. Secondary antibody conjugated to Alexa 546 (A11030) was purchased from Molecular probes (Eugene, Oreg.).

Worm and RNAi strains to discover aggregation and disaggregation activities. CL2006 (Link, Proc Natl Acad Sci USA 92: 9368, 1995) and N2 worm strains were obtained from the Caenorhabditis Genetics Center (Minneapolis, Minn.). The worms were grown at 20° C. To reduce gene expression we used previously described (Dillin et al., Science 298: 830, 2002) bacterial strains expressing dsRNA: empty vector (pAD12), daf-2 (pAD48) and daf-16 (pAD43). hsf-1 dsRNA expressing bacterial strain was from genomic RNAi library (J. Ahringer). Each RNAi bacteria colony was grown at 37° C. in LB with 100 μg/ml carbenicillin, and then seeded onto NG-carbenicillin plates supplemented with 100 mM IPTG.

Paralysis assay. Synchronous CL2006 worm populations were grown on (NG) plates containing 100 μg/ml carbenicillin, spotted with E. coli cultures expressing dsRNA as indicated. At first day of adulthood, 100 worms were placed on 10 plates (10 animals per plate). The plates were divided randomly to 5 sets (2 plates, 20 worms per set). The worms were tested every day for paralysis by tapping their noses with a platinum wire. Worms that moved their noses but failed to move their bodies were scored as “paralyzed”. To avoid scoring of old animals as paralyzed, paralysis assay terminated at day 12 of adulthood.

Organismal fractionation by Velocity sedimentation of Aβ₁₋₄₂. (i) Through sucrose gradient: Approximately twelve thousand CL2006 worms per sample were washed twice in M9 (RT), resuspended in 3001 PBS (pH7.4) and homogenized using a glass tissue grinder (885482, Kontes, Vineland, N.J.). The samples were spun in a desktop centrifuge (3000 rpm, 3 min, RT); post debris supernatants (PDS) were collected and incubated on ice for 30 min with 1% Sarkosyl. 10-60% sucrose step gradients in TNS (10 mM Tris-Cl pH 7.5, 150 mM NaCl, 1% Sarkosyl) were prepared in 2 ml TLS-55 tubes (300 μl each of 60, 40, 30, 20 and 10% sucrose). The worms preparations were laid on top of the gradients and the tubes were then spun at 55,000 rpm (gav=259,000 g) for 1 h at 4° C. in TLS-55 rotor (Beckman). Nine fractions of 220 μl each were collected from the top of the tube. (ii) Sedimentation: PDS were brought to 1% Sarkosyl, incubated on ice for the indicated time and then spun at 80,000 rpm for 1 h at 4° C. in a TLA120.2 rotor (gav=427,000 g). The pellets were then resuspended in PBS and protein loading buffer. Fractions were loaded on 12% Tris-Glycine PAA. Western blots were developed using an ECL system. For reprobing, PVDF membranes were stripped by incubation in 300 mM NaOH (5 min, RT), followed by neutralization by several rinses in TBST (10 mM Tris-Cl pH 7.5, 150 mM NaCl, 0.3% Tween-20).

In-vitro kinetic aggregation assay. Worms that were grown on RNAi bacterial strains as indicated, at the desired ages were washed twice with M9 and once more with PBS (RT). The worms were then resuspended in 3001 ice cold PBS, transferred to a 2 ml tissue grinder (885482, Kontes, Vineland, N.J.) and homogenized. Crude homogenates were spun in a desktop microfuge (3000 rpm, 3 min). Supernatants were transferred to new tubes and total protein concentrations were measured with BCA kit (Pierce, Rockford, Ill.). A stock solution of monomeric Aβ₁₋₄₀ peptide (300 μM) was prepared as previously described (38). Aβ₁₋₄₀ peptide was diluted to a final concentration of 20 μM in phosphate buffer (300 mM NaCl, 50 mM Na-phosphate, pH 7.4) containing ThT (20M). Post debris supernatants (PDS) were sonicated for 10 min in a water bath sonicator (FS60, Fisher Scientific, Pittsburg, Pa.) and added to the assay at a final total protein concentration of 0.3 mg/ml. Three aliquots (100 μl) of these solutions were transferred into wells of a 96-well microplate (Costar black, clear bottom) for each reaction. The plate was sealed and loaded into a Gemini SpectraMax EM fluorescence plate reader (Molecular Devices, Sunnyvale, Calif.), where it was incubated at 37° C. The fluorescence (excitation at 440 nm, emission at 485 nm) was measured from the bottom of the plate at 10 min intervals, with 5s of shaking before each reading. Half maximal fluorescence time points (t₅₀) were defined as the time point at which ThT fluorescence reached the middle between pre- and post-aggregation baselines. Fluorescence traces and t₅₀ values represent averages of at least three experiments.

Preparation of Worm Samples For Atomic Force Microscopy and Immuno-Em visualization. Worms were grown on RNAi bacterial strains as indicated. Post debris supernatants were prepared as described above and protein concentration was measured. Samples containing equal protein amounts were supplemented with ice-cold PBS to final volume of 1400 μl and loaded on top of a 300 μl 60% sucrose cushion in TNS. The tubes were spun for 20 min at 55,000 rpm (rotor TLS55, gav=259,000 g). 275 μl of sucrose and pellets were collected from the bottom of the tubes, supplemented with 900 μl ice cold PBS and were transferred to new tubes (final sucrose concentration 13.85%). The tubes were then spun at 80,000 rpm for 1 h at 4° C. in a TLA120.2 rotor (gav=427,000 g). The pellets were then resuspended in 50 μl purified water and subjected to atomic force microscopy or immuno-staining electron microscopy.

Atomic Force Microscopy (AFM). Aliquots (20 μl) were removed from the Aβ₁₋₄₀ aggregation solutions, from the dis-aggregation reactions (see below) or from worm preparations and placed on freshly cleaved mica (1×1 cm) mounted onto a metal sample holder. After 1 min, the solvent was absorbed into filter paper and the mica surface was washed thrice with 30 μl of water. Tapping-mode images were obtained on a MultiMode scanning probe microscope with a Nanoscope IIIa controller (Veeco, Woodbury, N.Y.). At least 9 images were analyzed for each sample. Fibril sizes were analyzed using the WSXM software (Nanotec Electronica S. L., Spain). All height scale bars represent 20 nm.

Immuno-staining electron microscopy (Immuno-EM). Aβ₁₋₄₂ aggregates were isolated from worms as described above. Preparations were adsorbed onto carbon covered copper grids and stained with anti Aβ6E10 antibody (5 gg/ml) and immuno-gold (10 nm) labeled secondary antibody and then counter-stained with 2% phosphotungsteic acid for negative staining, as previously described (39). No aggregate-specific immunostaining was observed without the 6E10 antibody (not shown). At least three independent pictures were analyzed for each sample. The pictures representing the highest signals for each sample are shown. 1 μm×1 μm areas of each picture were overlaid with a 100×100 nm grid and the number of gold particles in each square covering an aggregate surface was counted. Distribution histograms of the number of gold particles found within each square are shown.

In-vitro Aβ₁₋₄₀ disaggregation assay. Worms grown on RNAi bacterial strains as indicated to the desired ages were washed twice with M9 buffer and once more with PBS (RT). The worms were then resuspended in 3001 ice cold PBS, transferred to tissue grinder (885482, Kontes, Vineland, N.J.) and homogenized. Crude homogenates were spun in a desktop microfuge (3000 rpm, 3 min). Post Debris Supernatants (PDS) were transferred to new tubes and total protein concentrations were measured with a BCA kit (Pierce, Rockford, Ill.). A stock solution of monomeric Aβ₁₋₄₀ peptide (200 μM) was prepared as described in (38). Aβ₁₋₄₀ peptide (50 μM) was aggregated in phosphate buffer (150 mM NaCl, 50 mM Na-phosphate, pH 7.4) at 37° C. in a 1.5 ml reaction tube under constant agitation using an overhead shaker (20 rpm) for four days. Aβ₁₋₄₀ fibrils were sonicated for 30 min in a water bath sonicator (FS60, Fisher Scientific, Pittsburg, Pa.) and characterized by far UV CD spectroscopy and atomic force microscopy. Aβ₁₋₄₀ peptide was then diluted to a final concentration of 25 μM in phosphate buffer (150 mM NaCl, 50 mM Na-phosphate, pH 7.4) containing ThT (20 μM) and PDS worm homogenate (0.5 mg/ml) with or without proteasome inhibitor (10 μM Epoxymicin) or protease inhibitor cocktail as indicated. Three aliquots (100 μl) of each sample were incubated and ThT fluorescence was measured as described above.

Fluorescence data were fit to a single exponential decay function with a time constant T, allowing for a sloped post-disaggregation baseline (F (t)=mt+b+A exp(−t/τ)). Fitting residuals did not improve significantly by introducing a second exponential term. The first fluorescence data points (t<1 h), which showed a rapid decline in signal independent of disaggregation were ignored for subsequent analysis. τ-values are reported as averages+/−standard deviations determined from at least three independent samples

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for identifying a cellular modulator of a biological disaggregation activity of an animal comprising,
providing one or more polypeptide aggregate fibrils in solution,
contacting a biological sample from the animal with the polypeptide aggregate fibrils,
measuring a rate of fibril disappearance, and
identifying the modulator within the biological sample from the rate of fibril disappearance in the biological disaggregation activity.

2. The method of claim 1 further comprising forming the one or more polypeptide aggregate fibrils by transforming an amyloidogenic polypeptide or analog thereof into the one or more polypeptide aggregate fibrils.

3. The method of claim 2 further comprising transforming with seeding an amyloidogenic polypeptide or analog thereof into a polypeptide aggregate fibril.

4. The method of claim 1 wherein the one or more polypeptide aggregate fibrils are derived from a biological cell or tissue.

5. The method of claim 1 further comprising measuring the rate of fibril disappearance in the presence of at least one protease inhibitor.

6. The method of claim 1 wherein the biological disaggregation activity is denaturable.

7. The method of claim 1 wherein the cellular modulator is within an intracellular fraction.

8. The method of claim 1 wherein the cellular modulator is within an extracellular fraction.

9. The method of claim 1 wherein the polypeptide aggregate fibril is amyloid fibrils, alpha synuclein aggregate fibrils, or polyglutamine aggregate fibrils.

10. The method of claim 1 wherein the rate of fibril disappearance is measured by a reduction in amyloid fibril aggregates.

11. The method of claim 1 wherein the biological sample is from an animal with a mutation causing an aging program perturbation.

12. The method of claim 9 wherein the mutation causing the aging program perturbation is in daf-2, daf-16, or hsf-1 in Caenorhabditis elegans.

13. The method of claim 1 wherein the cellular modulator detoxifies polypeptide aggregate fibrils regulated by an aging program in the animal.

14. The method of claim 1 wherein the cellular modulator detoxifies protein aggregates not regulated by an aging program in the animal.

15. The method of claim 1 further comprising measuring the rate of fibril disappearance by measuring a reduction in fluorescence of fibril-binding environment-sensitive fluorophores.

16. The method of claim 15 wherein the fluorophores exhibit stronger fluorescence or wavelength-shifted fluorescence, or a combination thereof, when bound to fibrils compared to when the fluorophores are solvated in aqueous medium.

17. The method of claim 15 wherein the fluorophore is thioflavin T/S or Congo red.

18. The method of claim 1 further comprising measuring the rate of fibril disappearance by atomic force microscopy, electron microscopy or light microscopy.

19. The method of claim 1 further comprising measuring the rate of fibril disappearance by a decrease in anisotropy of fluorescently labeled solubilized amyloidogenic peptides.

20. The method of claim 1 further comprising measuring the rate of fibril disappearance by a decrease in turbidity or light scattering of the biological sample.

21. The method of claim 1 further comprising measuring the rate of fibril disappearance by appearance of monomers or low molecular weight oligomers of amyloid fibrils.

22. The method of claim 21 wherein measuring the appearance of monomers or low molecular weight oligomers is detected by gel electrophoresis, spectroscopically, chromomatographically, mass spectrometry or liquid chromatography mass spectrometry.

23. The method of claim 1 further comprising measuring a reduction of aggregates by SDS polyacrylamide gel electrophoresis followed by Western blotting.

24. The method of claim 1 further comprising measuring a reduction of aggregates by sucrose gradient centrifugation.

25. The method of claim 1 further comprising measuring a reduction of aggregates by native gel electrophoresis visualized by antibodies or amyloidophilic dyes.

26. A method for identifying a compound which modulates biological disaggregation activity in an animal comprising,
contacting a polypeptide aggregate fibril with the compound,
providing a biological sample from the animal in an amount selected to be effective to modulate biological disaggregation activity,
measuring a rate of fibril disappearance in the presence of the compound compared to a rate of fibril disappearance in the absence of the compound, and
detecting an effect of the compound on the biological disaggregation activity, effectiveness of the compound being indicative of an increase in biological disaggregation activity.

27. The method of claim 26 wherein the polypeptide aggregate fibril is a labeled polypeptide aggregate fibril prepared in vitro.

28. The method of claim 27 wherein the label is a fluorophore.

29. The method of claim 28 wherein the label is thioflavin T/S or Congo red.

30. The method of claim 26 wherein the polypeptide aggregate fibril is derived from a biological source.

31. The method of claim 26 further comprising contacting the polypeptide aggregate fibril with a seed.

32. The method of claim 26 wherein the biological sample is from an animal with an aging program perturbation.

33. The method of claim 26 wherein the biological sample is from the animal without an aging program perturbation.

34. The method of claim 26 wherein the polypeptide aggregate fibrils are aβ amyloid fibrils.

35. The method of claim 26 wherein the polypeptide aggregate fibrils are α-synuclein aggregates.

36. The method of claim 26 wherein the polypeptide aggregate fibrils are polyglutamine aggregates.

37. The method of claim 26 wherein the compound is a small chemical molecule, nucleic acid, antisense oligonucleotide, RNAi, ribozyme, oligosaccharide, antibody, polypeptide, or peptide mimetic.

38. The method of claim 26 wherein the compound is a chaperone, protease, or small heat shock protein.

39. The method of claim 26 wherein the biological disaggregation activity is denaturable.

40. The method of claim 26 wherein the rate of fibril disappearance is measured in the presence of at least one protease inhibitor.

41. The method of claim 26 wherein the cellular modulator is within an intracellular fraction.

42. The method of claim 26 wherein the cellular modulator is within an extracellular fraction.

43. The method of claim 26 wherein the rate of fibril disappearance is measured by a reduction in polypeptide aggregate fibril.

44. The method of claim 26 wherein the biological sample is from an animal with a mutation causing an aging program perturbation.

45. The method of claim 44 wherein the mutation causing the aging program perturbation is in daf-2, daf-16, or hsf-1 in Caenorhabditis elegans.

46. The method of claim 44 wherein the aging program perturbation in the animal results from an RNA interference screen.

47. A method for identifying a cellular modulator of a biological aggregation activity of an animal comprising,
providing an amyloidogenic polypeptide in solution,
contacting a biological sample with the amyloidogenic polypeptide,
measuring a rate of fibril appearance, and
identifying the modulator within the biological sample from the rate of fibril appearance in the biological aggregation activity.

48. The method of claim 47 further comprising measuring the rate of fibril appearance in the presence of at least one protease inhibitor.

49. The method of claim 47 wherein the biological aggregation activity is denaturable.

50. The method of claim 47 wherein the biological sample is an intracellular fraction.

51. The method of claim 47 wherein the biological sample is an extracellular fraction.

52. The method of claim 47 wherein the polypeptide aggregate fibril is amyloid fibrils, alpha synuclein aggregate fibrils, or polyglutamine aggregate fibrils.

53. The method of claim 52 wherein the rate of fibril appearance is measured by an increase in amyloid fibril aggregates.

54. The method of claim 47 wherein the biological sample is from an animal with a mutation causing an aging program perturbation.

55. The method of claim 54 wherein the mutation causing the aging program perturbation is in daf-2, daf-16, or hsf-1 in Caenorhabditis elegans.

56. The method of claim 47 wherein the cellular modulator detoxifies amyloidogenic polypeptides regulated by an aging program in the animal.

57. The method of claim 47 wherein the cellular modulator detoxifies amyloidogenic polypeptides not regulated by an aging program in the animal.

58. The method of claim 47 further comprising measuring the rate of fibril appearance by measuring an increase in the fluorescence of amyloid binding environment sensitive fluorophores.

59. The method of claim 58 wherein the fluorophores exhibit stronger fluorescence or wavelength-shifted fluorescence, or a combination thereof when bound to amyloid fibrils than when solvated in aqueous medium.

60. The method of claim 58 wherein the fluorophore is thioflavin T/S or Congo red.

61. The method of claim 47 further comprising measuring the rate of fibril appearance by atomic force microscopy, electron microscopy or light microscopy.

62. The method of claim 47 further comprising measuring the rate of fibril appearance by an increase in anisotropy of fluorescently labeled solubilized amyloidogenic peptides.

63. The method of claim 47 further comprising measuring the rate of fibril appearance by an increase in turbidity or light scattering of the biological sample.

64. The method of claim 47 further comprising measuring the rate of fibril appearance by disappearance of monomers or low molecular weight oligomers of amyloid fibrils.

65. The method of claim 64 wherein measuring the disappearance of monomers or low molecular weight oligomers is detected by gel electrophoresis, spectroscopically, chromomatographically, mass spectrometry or liquid chromatography mass spectrometry.

66. The method of claim 53 further comprising measuring an increase in aggregates by SDS polyacrylamide gel electrophoresis followed by Western blotting.

67. The method of claim 47 further comprising measuring an increase in aggregates by sucrose gradient centrifugation.

68. The method of claim 47 further comprising measuring an increase in aggregates by native gel electrophoresis visualized by antibodies or amyloidophilic dyes.

69. The method of claim 47 further comprising providing a quantity of seeds 1% or greater by weight in the solution to the amyloidogenic polypeptide in the solution to observe the biological aggregation activity.

70. A method for identifying a compound which modulates biological aggregation activity in a biological sample comprising,
providing an amyloidogenic polypeptide or analog thereof in a solution,
contacting the compound with the amyloidogenic polypeptide,
providing a biological sample from an animal in an amount selected to be effective to modulate biological aggregation activity,
measuring a rate of fibril appearance in the presence of the compound compared to a rate of fibril appearance in the absence of the compound, and
detecting an effect of the compound on the biological aggregation activity, effectiveness of the compound being indicative of an increase in biological aggregation activity.

71. The method of claim 70 wherein the biological sample is from an animal with an aging program perturbation.

72. The method of claim 70 wherein the biological sample is from the animal without an aging program perturbation.

73. The method of claim 70 wherein the polypeptide aggregate fibrils are aβ amyloid fibrils.

74. The method of claim 70 wherein the polypeptide aggregate fibrils are α-synuclein aggregates.

75. The method of claim 70 wherein the polypeptide aggregate fibrils are polyglutamine aggregates.

76. The method of claim 70 wherein the compound is a small chemical molecule, nucleic acid, antisense oligonucleotide, RNAi, ribozyme, oligosaccharide, antibody, polypeptide, or peptide mimetic.

77. The method of claim 70 wherein the compound is a chaperone, protease, or small heat shock protein.

78. The method of claim 70 wherein the biological aggregation activity is denaturable.

79. The method of claim 76 wherein the rate of fibril appearance is measured in the presence of protease inhibitors.

80. The method of claim 76 wherein the cellular modulator is within an intracellular fraction.

81. The method of claim 76 wherein the cellular modulator is within an extracellular fraction.

82. The method of claim 76 wherein the rate of fibril appearance is measured by an increase in polypeptide aggregate fibrils.

83. The method of claim 76 wherein the biological sample is from an animal with a mutation causing an aging program perturbation.

84. The method of claim 83 wherein the mutation causing the aging program perturbation is in daf-2, daf-16, or hsf-1 in Caenorhabditis elegans.

85. The method of claim 83 wherein the aging program perturbation results from an RNA interference screen.

86. The method of claim 76 further comprising providing a quantity of seeds 1% or greater by weight in the solution to the amyloidogenic polypeptide in the solution to observe the biological aggregation activity.
